(19) United States
(12) Patent Application Publication
(10) Pub. No.: US 2014/0248345 A1
(43) Pub. Date: Sep. 4, 2014

(54) PHARMACEUTICAL COMPOSITIONS OF COMBINATIONS OF DIPEPTIDYL PEPTIDASE-4 INHIBITORS WITH ATORVASTATIN

(71) Applicant: Merck Sharp & Dohme Corp., Rahway, NJ (US)

(72) Inventors: Sutthilug Sotthivirat, Lansdale, PA (US); Edward J. Fagan, Downingtown, PA (US); Katherine H. Chin, Pittsburgh, PA (US); Abraham B. Woldu, Souderton, PA (US); Charles DeLuca, Royersford, PA (US); Decheng Ma, Shanghai (CN); Walter R. Wasylaschuk, North Wales, PA (US); Bhagwant Rege, Germantown, MD (US)

(21) Appl. No.: 14/350,499

(22) PCT Filed: Oct. 20, 2012
(86) PCT No.: PCT/US2012/061304

§ 371 Date: Apr. 8, 2014

Related U.S. Application Data
(60) Provisional application No. 61/550,648, filed on Oct. 24, 2011.

(51) Int. Cl. A61K 31/4985; A61K 9/24; A61K 31/40

(52) U.S. Cl. CPC A61K 31/4985; A61K 31/40; A61K 9/209
USPC 424/464; 514/249

(57) ABSTRACT

This invention relates to a bilayer pharmaceutical compositions comprising fixed-dose combinations of a dipeptidyl peptidase-4 inhibitor and atorvastatin, or a pharmaceutically acceptable salt thereof, methods of preparing such pharmaceutical compositions, and methods of treating Type 2 diabetes with such pharmaceutical compositions.

### BACKGROUND OF THE INVENTION

Type 2 diabetes is a chronic and progressive disease arising from a complex pathophysiology involving the dual endocrine defects of insulin resistance and impaired insulin secretion. The treatment of Type 2 diabetes typically begins with diet and exercise, followed by oral antidiabetic monotherapy. Many patients with type 2 diabetes are considered to be at high risk for coronary artery disease and associated co-morbidities. Coronary artery disease is a multifactorial disease in which the incidence and severity are affected by a myriad of factors such as lipid profile, presence of diabetes and the sex of the patient. In order to meaningfully reduce the risk of coronary artery disease, it is crucial to manage the entire risk spectrum. Treatment with cholesterol synthesis inhibitors in patients with and without coronary heart disease, including coronary artery disease, reduces the risk of cardiovascular morbidity and, mortality.

For many patients at risk of a coronary heart disease event due to existing diabetes, treatment with both an anti-diabetic agent and a cholesterol synthesis inhibitor is recommended. However, co-prescription of an oral anti-diabetic drug and an oral cholesterol synthesis inhibitor may result in treatment regimens that are complex and difficult for many patients to follow. Combining an anti-diabetic agent and a cholesterol synthesis inhibitor into a single tablet provides a potential means of delivering combination therapy without adding to the complexity of patients' daily regimens. Such formulations have been well accepted in other disease indications, such as hypertension (HYZAAR™ which is a combination of losartan potassium and hydrochlorothiazide) and cholesterol lowering (VYTORIN™ which is a combination of simvastatin and ezetimibe). The selection of effective and well-tolerated treatments is a key step in the design of a combination tablet. Moreover, it is essential that the components have complementary mechanisms of action and compatible pharmacokinetic profiles. Examples of marketed combination tablets containing two oral antidiabetic agents include Glucovance™ (metformin and glyburide), Avandamet™ (metformin and rosiglitazone), and Metaglip™ (metformin and glipizide).

Currently, sitagliptin and atorvastatin are available as separate tablets for the treatment of type 2 diabetes, hypercholesterolemia and hyperlipidemia. This invention provides a pharmaceutical composition comprising sitagliptin, or a pharmaceutically acceptable salt thereof, and atorvastatin, or a pharmaceutically acceptable salt thereof, as a fixed dose combination in a single bilayer tablet with enhanced chemical and physical stability, and minimal degradation, of both active ingredients. This invention further provides a pharmaceutical composition comprising sitagliptin, or a pharmaceutically acceptable salt thereof, and atorvastatin, or a pharmaceutically acceptable salt thereof, as a fixed dose combination in a single bilayer tablet for superior efficacy, patient convenience and improved compliance for the treatment of type 2 diabetes, hypercholesterolemia and hyperlipidemia.

Atorvastatin calcium is a selective, competitive inhibitor of the enzyme 3-hydroxy-3-methyl glutaryl coenzyme A reductase (HMG-CoA reductase), and can be used to lower blood cholesterol by reducing the production of cholesterol by the liver. The conversion of 3-hydroxy-3-methylglutaryl-coenzyme A (HMG-CoA) to mevalonate is an early rate-limiting step in the cholesterol biosynthetic pathway. This step is catalyzed by the enzyme HMG-CoA reductase. Atorvastatin calcium inhibits HMG-CoA reductase from catalyzing this conversioh, and is therefore a potent lipid lowering agent useful for the prevention and treatment of those conditions caused or exacerbated by high levels of cholesterol.

Atorvastatin calcium is commercially available for the treatment of primary hypercholesterolemia, dysbetalipoproteinemia, and homozygous familial hypercholesterolemia in the U.S. and elsewhere under the trademark name LIPITOR®. LIPITOR® is also indicated to reduce the risk of myocardial infarction in adult hypertensive patients without clinically evident coronary heart disease, but with at least three additional risk factors for coronary heart disease, including but not limited to age>55 years, male sex, smoking, type 2 diabetes and ratio of plasma total cholesterol to HDL-cholesterol>6. LIPITOR® is also indicated to reduce the risk of myocardial infarction and stroke in adult patients with type 2 diabetes mellitus and hypertension without clinically evident coronary heart disease, but with other risk factors such as age>55 years, retinopathy, albuminaria or smoking. In particular, in patients at high risk of coronary events because of existing coronary heart disease, diabetes and peripheral vessel disease, a history of stroke or other cerebrovascular disease, LIPITOR® is indicated to reduce the risk of total mortality by reducing coronary heart disease deaths, by reducing the risk of non-fatal myocardial infarction and stroke, and by reducing the need for coronary and non-coronary revascularization procedures in patients at high risk of coronary events.

The active pharmaceutical ingredient in LIPITOR® is atorvastatin present in a salt form, described in the Physician's Desk Reference as having the chemical name R-(R*,R*)]-2-(4-fluorophenyl)-β,δ-dihydroxy-5-(1-methylethyl)-3-phenyl-4-[(phenylamino) carbonyl]-1H-pyrrole-1-heptanoic acid, calcium salt (2:1) trihydrate (also known as atorvastatin calcium trihydrate). The active pharmaceutical ingredient in LIPITOR® has the following structural formula:

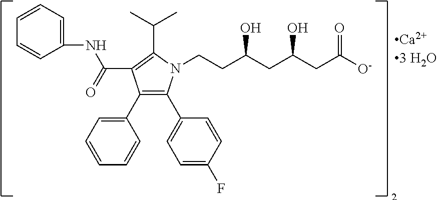

Atorvastatin calcium is disclosed in U.S. Pat. No. 5,273,995; amorphous and crystalline forms of atorvastatin calcium are disclosed in U.S. Pat. Nos. 5,969,156; 6,121,461; 6,605,729; and PCT Publications WO2006/011041, WO2004/050618, WO2003/070702, WO2002/041834, and WO2001/036384; and stable oral formulations of atorvastatin calcium are disclosed in U.S. Pat. Nos. 5,686,104 and 6,126,971; all of which are incorporated by reference herein.

Atorvastatin is susceptible to heat, moisture, low pH environment, and light. In an acidic environment, in particular, the hydroxy acid moiety of atorvastatin will degrade to lactone. In addition, the hydroxy acid will decompose rapidly when exposed to UV or fluorescent light.

When packaged in the form of tablets, powders, granules, or within capsules, atorvastatin may be further destabilized by contact with the molecular moieties of other excipients. Because pharmaceutical dosage excipients such as binders, diluents, anti-adherents, surfactants and the like may adversely interact with atorvastatin, a need exists for improved stabilizing means for effective pharmaceutical dosages.

Dipeptidyl peptidase-4 (DPP-4) inhibitors represent a class of agents that are being developed for the treatment or improvement in glycemic control in patients with Type 2 diabetes. Specific DPP-4 inhibitors currently in clinical trials for the treatment of Type 2 diabetes include sitagliptin phosphate (MK-0431), vildagliptin (LAF-237), saxagliptin (BMS-47718), alogliptin, carmegliptin, melogliptin, dutogliptin, denagliptin, linagliptin, P93/01 (Prosidion), SYR322 (Takeda), GSK 823093, Roche 0730699, TS021 (Taisho), E3024 (Eisai), and PIX-1149 (Phenomix). For example, oral administration of vildagliptin or sitagliptin to human Type 2 diabetics has been found to reduce fasting glucose and postprandial glucose excursion in association with significantly reduced HbA_{1c} levels. For reviews on the application of DPP-4 inhibitors for the treatment of Type 2 diabetes, reference is made to the following publications: (1) H.-U. Demuth, et al., “Type 2 diabetes—Therapy with dipeptidyl peptidase IV inhibitors, Biochim. Biophys. Acta, 1751: 33-44 (2005) and (2) K. Augustyns, et al., “Inhibitors of proline-specific dipeptidyl peptidases: DPP IV inhibitors as a novel approach for the treatment of Type 2 diabetes,” Expert Opin. Ther. Patents, 15: 1387-1407 (2005).

Sitagliptin phosphate having structural formula I below is the dihydrogen phosphate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine.

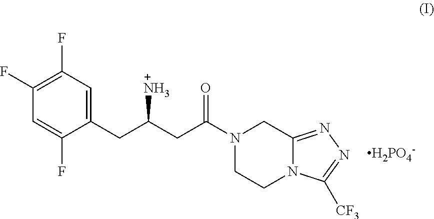

In one embodiment, sitagliptin phosphate is in the form of a crystalline anhydrate or monohydrate. In a class of this embodiment, sitagliptin phosphate is in the form of a crystalline monohydrate. Sitagliptin free base and pharmaceutically acceptable salts thereof are disclosed in U.S. Pat. No. 6,699,871, the contents of which are hereby incorporated by reference in their entirety. Crystalline sitagliptin phosphate monohydrate is disclosed in international patent publication WO 2005/0031335 published on Jan. 13, 2005. For a review on sitagliptin phosphate (MK-0431) including its synthesis and pharmacological properties, reference is made to the following publications: (1) C. F. Deacon, “MK-431,” Curr. Opin. Invest. Drugs, 6: 419-426 (2005) and (2) “MK-0431”, Drugs of the Future,” 30: 337-343 (2005).

Vildagliptin (LAF-237) is the generic name for (S)-1-[(3-hydroxy-1-adamantyl)amino]acetyl-2-cyano-pyrrolidine having structural formula III. Vildagliptin is specifically disclosed in U.S. Pat. No. 6,166,063, the contents of which are hereby incorporated by reference in their entirety.

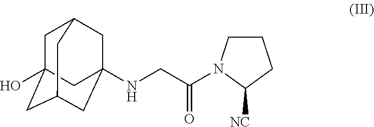 [SMILES: N#C[C@@H]1CCCN1C(=O)CNC12CC3CC(CC(O)(C3)C1)C2]

Saxagliptin (BMS-47718) is a methanoprolinenitrile of structural formula IV below. Saxagliptin is specifically disclosed in U.S. Pat. No. 6,395,767, the contents of which are hereby incorporated by reference in their entirety.

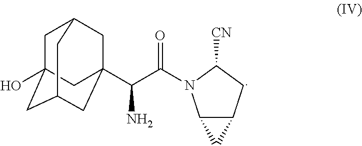 [SMILES: N#C[C@@H]1C[C@@H]2C[C@@H]2N1C(=O)[C@@H](N)C12CC3CC(CC(O)(C3)C1)C2]

Alogliptin (SYR-322) is a DP-IV inhibitor under investigation for the treatment of type 2 diabetes of structural formula V below:

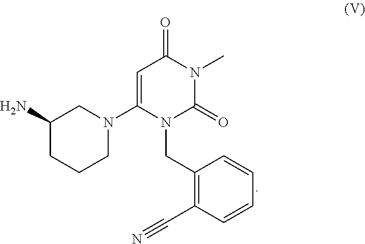 [SMILES: Cn1c(=O)cc(N2CCC[C@@H](N)C2)n(Cc2ccccc2C#N)c1=O]

Other DP-IV inhibitors useful in the formulation of the present invention include, but are not limited to: alogliptin, carmegliptin, melogliptin, dutogliptin, denagliptin, linagliptin, saxagliptin and vildagliptin.

The present invention provides pharmaceutical compositions comprised of a fixed-dose combination of a dipeptidyl peptidase-4 inhibitor (DPP-4 inhibitor), or a pharmaceutically acceptable salt thereof, and atorvastatin, or a pharmaceutically acceptable salt thereof, in a bilayer tablet.

The present invention further provides for pharmaceutical compositions of a fixed-dose combination of a dipeptidyl peptidase-4 inhibitor (DPP-4 inhibitor), or a pharmaceutically acceptable salt thereof, and atorvastatin, or a pharmaceutically acceptable salt thereof, which are prepared by dry and/or wet processing methods. The pharmaceutical compositions of the present invention provide for immediate release of the two active pharmaceutical ingredients. In one embodiment the pharmaceutical compositions of the present invention are in the dosage form of a tablet, and, in particular, a film-coated tablet. The present invention further provides for a fixed dose combination of

The present invention also provides a process to prepare pharmaceutical compositions of a fixed-dose combination of a DPP-4 inhibitor, or a pharmaceutically acceptable salt thereof, and atorvastatin, or a pharmaceutically acceptable salt thereof, by dry and wet processing methods. The dry processing methods include dry compression and dry granulation, and the wet processing methods include wet granulation, such as fluid bed granulation and high-shear granulation. In one embodiment, the DPP-4 inhibitor layer is prepared by direct dry compression, and the atorvastatin layer is prepared by dry granulation. Individual layers are subsequently compressed together using a bilayer press and optionally film coated.

Another aspect of the present invention provides methods for the treatment of Type 2 diabetes and hypercholesterolemia by administering to a patient in need of such treatment a therapeutically effective amount of a pharmaceutical composition of the present invention.

These and other aspects will become readily apparent from the detailed description which follows.

### SUMMARY OF THE INVENTION

The present invention is directed to novel pharmaceutical compositions comprising fixed dose combinations of a dipeptidyl peptidase-4 inhibitor (DPP-4 inhibitor), or a pharmaceutically acceptable salt thereof, and atorvastatin, or pharmaceutically acceptable salt thereof, methods of preparing such pharmaceutical compositions, and methods of treating Type 2 diabetes and hypercholesterolemia with such pharmaceutical compositions.

The invention is further directed to pharmaceutical compositions comprising fixed-dose combinations of sitagliptin, or a pharmaceutically acceptable salt, hydrate and/or solvate thereof, and atorvastatin, or a pharmaceutically acceptable salt, hydrate and/or solvate thereof. In particular, the invention is directed to pharmaceutical compositions comprising fixed-dose combinations of sitagliptin phosphate, or a hydrate or solvate thereof, and atorvastatin calcium, or a hydrate or solvate thereof.

### DETAILED DESCRIPTION OF THE INVENTION

One aspect of the present invention is directed to dosage forms for the medicinal administration of a fixed-dose combination of a dipeptidyl peptidase-4 inhibitor (DPP-4 inhibitor) and atorvastatin. Such dosage forms may be in the powder or solid format including, but not limited to, tablets, capsules, and sachets.

The present invention provides for fixed-dose combinations in the form of a bilayer tablet of a dipeptidyl peptidase-4 inhibitor (such as sitagliptin), or a pharmaceutically acceptable salt thereof, and atorvastatin, or a pharmaceutically acceptable salt thereof, which may have the benefit of improved chemical stability and increased dissolution of the active ingredients relative to the corresponding monolithic tablet.

It is known that statins form an intramolecular lactone ring, and that the lactone has been considered to be a prodrug that converts to the active hydroxy acid form. It is also known that the physicochemical properties of the lactone and hydroxy acid forms of statins impacts their formulation and biologic performance. It has been found that when atorvastatin exists as the free acid form it is 15 times more soluble than as the lactone form. (Kearney et al., Pharmaceutical Research, Vol. 10, No. 10, pp. 1461-1465 (1993)). To increase the solubility, bioavailability and drug product purity of atorvastatin, it is desirable to prevent the lactone ring formation in the atorvastatin layer of the bilayer tablet.

The bilayer tablet of the present invention comprising sitagliptin and atorvastatin, or pharmaceutically acceptable salts thereof, is expected to result in a decrease in the atorvastatin lactone formation, and a decrease in the sitagliptin—atorvastatin adduct byproduct relative to a monolithic tablet comprising sitagliptin and atorvastatin, or pharmaceutically acceptable salts thereof. Further, the bilayer tablet of the present invention comprising sitagliptin and atorvastatin, or pharmaceutically acceptable salts thereof, resulted in an increase in the dissolution rate of atorvastatin layer. An increase in the dissolution rate of atorvastatin is associated with an increase in bioavailability.

The formulation of sitagliptin and atorvastatin of the present invention resulted in several unexpected benefits. It was unexpectedly found that the use of SiO₂ (including silicified microcrystalline cellulose or Prosolv®) in the sitagliptin layer of the bilayer tablet resulted in a decrease in the formation of the atorvastatin lactone in the atorvastatin layer. It was further unexpectedly found that increasing the amount of dibasic calcium phosphate in the sitagliptin layer resulted in the faster dissolution of the atorvastatin layer.

Another unexpected benefit resulted from the substitution of mannitol for lactose as the diluent in the atorvastatin layer. It was unexpectedly found that the use of mannitol in the atorvastatin layer resulted in faster dissolution of the atorvastatin layer relative to the dissolution with lactose as the diluent. It was also unexpectedly found that the use of mannitol as the diluent in the atorvastatin layer lead to a decrease in the amount of atorvastatin lactone fcirmed in the atorvastatin layer relative to the amount of lactone formed using lactose as the diluent.

Finally, the bilayer tablets of the present invention comprising a DPP-4 inhibitor, such as sitagliptin, and atorvastatin, or pharmaceutically acceptable salts thereof, have the additional benefit of providing a stable fixed dose combination for storage.

As used herein with respect to the compositions of this invention, the term “atorvastatin” encompasses R(R*,R*)]-2-(4-fluorophenyl)-β,δ-dihydroxy-5-(1-methylethyl)-3-phenyl-4-[(phenylamino)carbonyl]-1H-pyrrole-1-heptanoic acid and pharmaceutically acceptable salts thereof, and encompasses all physical forms of the aforementioned forms, including but not limited to hydrate, solvate, amorphous and crystalline forms.

Physical form refers to the spatial order or packing of molecules, and includes for example solid crystalline, liquid-crystalline, non-crystalline and amorphous physical forms of atorvastatin and mixtures thereof. Any anhydrate or hydrate forms of atorvastatin are also encompassed. The term “atorvastatin” is not intended to be limited to the free acid, a particular pharmaceutically acceptable salt, or any particular physical form (e.g., crystalline or amorphous) unless specified otherwise. The term atorvastatin includes, but is not limited to, atorvastatin calcium salt, atorvastatin calcium salt trihydrate, atorvastatin calcium salt (2:1), atorvastatin calcium salt (2:1) trihydrate), as specified. Aside from determining the molar amount of atorvastatin in a molar ratio, when the term “atorvastatin” is combined with an express description of form, then that particular form of atorvastatin is intended (for example, “atorvastatin free acid,” “atorvastatin calcium”, “amorphous atorvastatin”, “amorphous atorvastatin calcium”, or “amorphous atorvastatin calcium trihydrate”). Atorvastatin free acid is shown below as structural formula I:

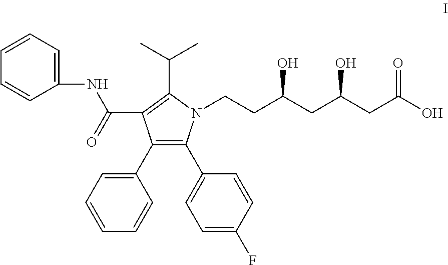 [SMILES: CC(C)c1c(C(=O)Nc2ccccc2)c(-c2ccccc2)c(-c2ccc(F)cc2)n1CC[C@@H](O)C[C@@H](O)CC(=O)O]

In addition to the crystalline polymorphs of atorvastatin calcium discussed supra, various salts of atorvastatin have been described, for example, in U.S. Pat. Nos. 4,681,893 and 5,273,995; and in WO2005/105738, WO2005/115980, WO2006/117761, WO2007/132472 and WO2007/063551; and crystalline forms of atorvastatin free acid are described in US2007-0276027; the contents of each which are herein incorporated by reference in their entirety. Additionally, formulations of atorvastatin have been disclosed in U.S. Pat. No. 5,686,104 and U.S. Pat. No. 7,790,197.

As used herein, “alkalizing additive” means an additive selected from sodium bicarbonate and L-arginine. As used herein, the term “alkalizing agent” also means calcium carbonate.

The term “active agent” as used herein means an agent having pharmaceutical activity, as distinguished from an excipient or drug carrier.

Pharmaceutically acceptable salts of atorvastatin are preferred for use in all embodiments of this invention. Pharmaceutically acceptable salts of atorvastatin within the scope of the invention are those derived from bases such as sodium hydroxide, potassium hydroxide, lithium hydroxide, calcium hydroxide, 1-deoxy-2-(methylamino)-D-glucitol, magnesium hydroxide, zinc hydroxide, aluminum hydroxide, ferrous or ferric hydroxide, ammonium hydroxide or organic amines such as N-methylglucamine, choline, arginine and the like. Suitable pharmaceutically acceptable salts of atorvastatin include but are not limited to pharmaceutically acceptable metal salts, particularly alkali metal salts such as lithium, sodium or potassium salts, and alkaline earth metal salts such as magnesium or calcium salts. In one embodiment of the present invention, the salt is the sodium hydroxide or sodium salt. In another embodiment of the present invention, the salt is the calcium hydroxide or calcium salt.

Atorvastatin calcium salt or atorvastatin calcium salt (2:1) is the most preferred salt form. Additionally, in all embodiments of the invention atorvastatin is preferably present in the compositions in a pharmaceutically acceptable salt form as described above, wherein the salt is in either a crystalline or amorphous physical form. Crystalline or amorphous atorvastatin calcium salt or crystalline or amorphous atorvastatin calcium salt (2:1) is more preferred, and amorphous atorvastatin calcium salt or atorvastatin calcium salt (2:1) is most preferred. In one embodiment of the present invention, atorvastatin is atorvastatin calcium salt (2:1) trihydrate. In another embodiment of the present invention, atorvastatin is crystalline atorvastatin calcium salt (2:1) trihydrate. In another embodiment of the present invention, atorvastatin is crystalline atorvastatin calcium salt (2:1) trihydrate crystal form I.

Examples of pharmaceutically acceptable salts of atorvastatin and various solvate, hydrate and physical forms thereof are described in many publications, including but not limited to, WO2005/115980, WO2005/105738 and U.S. Pat. Nos. 4,681,893 and 5,273,995, each of which are herein incorporated by reference in their entirety.

Atorvastatin can exist in crystalline, liquid crystalline and non-crystalline and amorphous forms. It is known that the amorphous forms in a number of drugs exhibit different dissolution characteristics and in some cases different bioavailability patterns. For some therapeutic indications one bioavailability pattern may be favored over another. Variations in dissolution rates can make it advantageous to produce atorvastatin formulations in either crystalline or amorphous forms. Non-crystalline and crystalline forms of atorvastatin free acid are described, for example, in US Patent Application Publication US2007/0276027.

Formulations of the tablets of the present invention are made in accordance with methods that are standard in the art (see, e.g., Remington's Pharmaceutical Sciences, 16^{th} ed., A Oslo editor, Easton, Pa. (1980)). Drug combinations such as those claimed in the instant invention will typically be prepared in admixture with conventional excipients. Coating layer(s) may be applied as well using standard coating techniques.

In another embodiment of the present invention, there is an oxygen bather film coating. This coating prevents oxygen permeation into the core tablets, which contain at least one active ingredient (e.g. atorvastatin) that is sensitive to molecular oxygen (O₂). The film coating can protect the active ingredient from the effects of environmental O₂.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient(s), and pharmaceutically acceptable excipients.

There are a wide variety of excipients known in the pharmaceutical formulation art. According to the desired properties of the pharmaceutical composition, any number of ingredients may be selected, alone or in combination, based upon their known uses in preparing tablet compositions.

The pharmaceutical compositions of the present invention optionally include one or more excipients selected from binders, disintegrants, lubricants, surfactants, diluents, anti-oxidants, compression aids, glidants, flavors, flavor enhancers, sweeteners, and preservatives, and combinations thereof. The quantity of each excipient is expressed as a weight percentage of the first or second layer of the bilayer tablet, corresponding to the layer that the excipient is in. Each of the weight percentage amounts noted for each excipient can be combined with any weight percentage amount noted for one or more of the other excipients, and all such combinations are encompassed within the scope of this invention.

In a particular aspect of the present invention, the pharmaceutical compositions in the form of a bilayer tablet comprise: (a) a first layer comprising a dipeptidyl peptidase-4 inhibitor, or a pharmaceutically acceptable salt thereof; and (b) a second layer comprising atorvastatin. In one embodiment of the present invention, the first bilayer additionally comprises one or more excipients selected from the group consisting of: (i) a diluent; (ii) a disintegrant; and (iii) a lubricant. In another embodiment of the present invention, the second bilayer additionally comprises one or more excipients selected from the group consisting of (i) a diluent, (ii) an anti-oxidant; (iii) a binding agent; and (iv) a lubricant. In another embodiment of the present invention, the pharmaceutical compositions may also contain one or more surfactants or wetting agents; and one or more antioxidants.

In another embodiment of this aspect of the invention, the DPP-4 inhibitor is selected from the group consisting of sitagliptin, vildagliptin, saxagliptin, P93/01, SYR322, GSK 823093, Roche 0730699, TS021, E3024, and PHX-1149. In a class of this embodiment the DPP-4 inhibitor is alogliptin, carmegliptin, melogliptin, dutogliptin, denagliptin, linagliptin, sitagliptin, vildagliptin, or saxagliptin. In a subclass of this class, the DPP-4 inhibitor is sitagliptin. A preferred pharmaceutically acceptable salt of sitagliptin is the dihydrogen phosphate salt of structural formula I above (sitagliptin phosphate). A preferred form of the sitagliptin dihydrogen phosphate salt is the crystalline monohydrate (sitagliptin phosphate monohydrate) disclosed in WO 2005/0031335.

The preparation of sitagliptin and pharmaceutically acceptable salts thereof is disclosed in U.S. Pat. No. 6,699,871, the contents of which are herein incorporated by reference in their entirety. The preparation of sitagliptin phosphate monohydrate is disclosed in international patent publication WO 2005/0031335 published on Jan. 13, 2005, the contents of which are herein incorporated by reference in their entirety.

The dosage strength of the DPP-4 inhibitor for incorporation into the pharmaceutical compositions of the present invention is an amount from about 1 milligram to about 250 milligrams of the active moiety. A preferred dosage strength of the DPP-4 inhibitor is an amount from about 25 milligrams to about 200 milligrams of the active moiety. Discrete dosage strengths are the equivalent of 25, 50, 75, 100, 150, and 200 milligrams of the DPP-4 inhibitor active moiety. By “active moiety” is meant the free base form of the DPP-4 inhibitor as an anhydrate.

The unit dosage strength of sitagliptin free base anhydrate (active moiety) for inclusion into the fixed-dose combination pharmaceutical compositions of the present invention is 25, 50, 75, 100, 150, or 200 milligrams. An equivalent amount of sitagliptin phosphate monohydrate to the sitagliptin free base anhydrate is used in the pharmaceutical compositions, namely, 32.13, 64.25, 96.38, 128.5, 192.75, and 257 milligrams, respectively. A preferred dosage strength of sitagliptin is 50 or 100 milligrams. Another preferred dosage strength of sitagliptin is 100 milligrams. Another preferred dosage strength of sitagliptin is 50 milligrams.

The percentage of atorvastatin, and optional additional active agent(s) in the compositions of the present invention may be varied, it being necessary that it should constitute a proportion such that a suitable dosage shall be obtained. The unit dosage strength of atorvastatin free acid (active moiety) for incorporation into the pharmaceutical compositions of the present invention is an amount from about 1 milligram to about 100 milligrams. The atorvastatin is preferably administered to the patient once a day at a unit dosage strength of 5, 10, 20, 40, or 80 mg per day, on an atorvastatin free acid weight basis. An equivalent amount of atorvastatin calcium to the atorvastatin free acid (or active moiety) is used in the pharmaceutical compositions, namely, 5.17 mg, 10.34 mg, 20.68 mg, 41.36 mg and 82.72 mg, respectively. A preferred dosage strength of atorvastatin is an amount from about 5 milligrams to about 80 milligrams of the active moiety. Discrete dosage strengths are the equivalent of 5, 10, 20, 40, and 80 milligrams of atorvastatin. Another preferred dosage strength of atorvastatin is 5, 10, 20, 40 or 80 milligrams of atorvastatin. Another preferred dosage strength of atorvastatin is 10, 20, 40 or 80 milligrams of atorvastatin. Another preferred dosage strength of atorvastatin is 10, 20, or 40 milligrams of atorvastatin. These unit dosage strengths of atorvastatin represent the dosage strengths approved in the U.S. for marketing to treat Type 2 diabetes. In each particular case, the doses are determined by a physician in accordance with the factors distinctive to the patient to be treated, such as age, weight, general state of health and other characteristics which can influence the efficacy of the atorvastatin, and optional additional active agent(s).

Specific embodiments of dosage strengths for sitagliptin and atorvastatin in the fixed-dose combinations of the present invention are the following:
(1) 100 milligrams of sitagliptin (equivalent to 128.5 milligrams of sitagliptin phosphate monohydrate) and 5 milligrams atorvastatin (equivalent to 5.17 milligrams of atorvastatin sodium or atorvastatin sodium);
(2) 100 milligrams of sitagliptin (equivalent to 128.5 milligrams of sitagliptin phosphate monohydrate) and 10 milligrams atorvastatin (equivalent to 10.34 milligrams of atorvastatin calcium);
(3) 100 milligrams of sitagliptin (equivalent to 128.5 milligrams of sitagliptin phosphate monohydrate) and 20 milligrams atorvastatin (equivalent to 20.68 milligrams of atorvastatin calcium);
(4) 100 milligrams of sitagliptin (equivalent to 128.5 milligrams of sitagliptin phosphate monohydrate) and 40 milligrams atorvastatin (equivalent to 41.36 milligrams of atorvastatin calcium);
(5) 100 milligrams of sitagliptin (equivalent to 128.5 milligrams of sitagliptin phosphate monohydrate) and 80 milligrams atorvastatin (equivalent to 82.72 milligrams of atorvastatin calcium);
(6) 50 milligrams of sitagliptin (equivalent to 64.25 milligrams of sitagliptin phosphate monohydrate) and 5 milligrams atorvastatin (equivalent to 5.17 milligrams of atorvastatin calcium);
(7) 50 milligrams of sitagliptin (equivalent to 64.25 milligrams of sitagliptin phosphate monohydrate) and 10 milligrams atorvastatin (equivalent to 10.34 milligrams of atorvastatin calcium);
(8) 50 milligrams of sitagliptin (equivalent to 64.25 milligrams of sitagliptin phosphate monohydrate) and 20 milligrams atorvastatin (equivalent to 20.68 milligrams of atorvastatin calcium);
(9) 50 milligrams of sitagliptin (equivalent to 64.25 milligrams of sitagliptin phosphate monohydrate) and 40 milligrams atorvastatin (equivalent to 41.36 milligrams of atorvastatin calcium), and
(10) 50 milligrams of sitagliptin (equivalent to 64.25 milligrams of sitagliptin phosphate monohydrate) and 80 milligrams atorvastatin (equivalent to 82.72 milligrams of atorvastatin calcium).

The pharmaceutical compositions of the present invention are prepared by dry/or and wet processing methods. In one embodiment the atorvastatin layer is prepared by dry processing methods. In a class of this embodiment, the atorvastatin layer is prepared by dry granulation methods. In another class of this embodiment, the atorvastatin layer is prepared by direct compression or direct compression blend. In another embodiment of the present invention, the atorvastatin layer is prepared by fluid bed granulation.

In another embodiment the DPP-4 layer is prepared by dry processing methods. In a class of this embodiment, the DPP-4 layer is prepared by direct compression or direct compression blend.

The pharmaceutical compositions obtained by dry and/or wet processing methods may be compressed into tablets using a bilayer press, encapsulated, or metered into sachets, and optionally film coated.

In another embodiment of the present invention, the pharmaceutical compositions may contain one or more lubricants. Examples of lubricants include magnesium stearate, magnesium stearate (non-bovine), calcium stearate, stearic acid, sodium stearyl fumarate, hydrogenated castor oil, and mixtures thereof. In one embodiment, the lubricant is magnesium stearate or sodium stearyl fumarate, or a mixture thereof. In another embodiment, the lubricant is a mixture of magnesium stearate and sodium stearyl fumarate. In another embodiment, the lubricant is magnesium stearate. In another embodiment, the lubricant is sodium stearyl fumarate.

In another embodiment of the present invention, the pharmaceutical compositions may contain one or more glidants. Examples of glidants include silicon dioxide, calcium phosphate tribasic, magnesium silicate, and talc. In one embodiment of the present invention, the glidant is selected from silicon dioxide, calcium phosphate tribasic, magnesium silicate, and talc. In another embodiment of the present invention, the glidant is silicon dioxide (SiO₂).

In another embodiment of the present invention, the pharmaceutical composition contains about 0-5% of a glidant. In a class of this embodiment, the glidant is silicon dioxide. In another embodiment of the present invention, the pharmaceutical composition contains about 0-2% of a glidant. In a class of this embodiment, the glidant is silicon dioxide. In another embodiment of the present invention, the pharmaceutical Composition contains about 0-1% of a glidant. In a class of this embodiment, the glidant is silicon dioxide. In another embodiment of the present invention, the pharmaceutical composition contains about 0.1-1% of a glidant. In a class of this embodiment, the glidant is silicon dioxide. In another embodiment of the present invention, the pharmaceutical composition contains about 0.25-1% of a glidant. In a class of this embodiment, the glidant is silicon dioxide.

In another embodiment of the present invention, the first layer of the pharmaceutical composition (the DPP-4/sitagliptin layer) contains about 0 to 5% of a glidant. In class of this embodiment, the glidant is silicon dioxide. In another embodiment of the present invention, the first layer of the pharmaceutical composition (the DPP-4/sitagliptin layer) contains about 0 to 2% of a glidant. In class of this embodiment, the glidant is silicon dioxide. In another embodiment of the present invention, the first layer of the pharmaceutical composition (the DPP-4/sitagliptin layer) contains about 0 to 1% of a glidant. In class of this embodiment, the glidant is silicon dioxide. In another embodiment of the present invention, the first layer of the pharmaceutical composition (the DPP-4/sitagliptin layer) contains about 0.1 to 1% of a glidant. In class of this embodiment, the glidant is silicon dioxide. In another embodiment of the present invention, the first layer of the pharmaceutical composition (the DPP-4/sitagliptin layer) contains about 0.25 to 1% of a glidant. In class of this embodiment, the glidant is silicon dioxide. In another embodiment of the present invention, the first layer of the pharmaceutical composition (the DPP-4/sitagliptin layer) contains about 0.25%, 0.5%, 1% or 2% of a glidant. In class of these embodiments, the glidant is silicon dioxide.

In another embodiment of the present invention, the second layer of the pharmaceutical composition (the atorvastatin layer) contains about 0 to 5% of a glidant. In another embodiment of the present invention, the second layer of the pharmaceutical composition (the atorvastatin layer) contains about 0 to 2% of a glidant. In another embodiment of the present invention, the second layer of the pharmaceutical composition (the atorvastatin layer) contains about 0 to 1% of a glidant. In another embodiment of the present invention, the second layer of the pharmaceutical composition (the atorvastatin layer) contains about 0.1 to 1% of a glidant. In another embodiment of the present invention, the second layer of the pharmaceutical composition (the atorvastatin layer) contains about 0.25 to 1% of a glidant. In another embodiment of the present invention, the second layer of the pharmaceutical composition (the atorvastatin layer) contains about 0.25%, 0.5%, 1%, 2% or 4% of a glidant. In another embodiment of the present invention, the second layer of the pharmaceutical composition (the atorvastatin layer) contains about 0.26%, 0.52%, 1.04%, 2.08 or 4.16% of a glidant. In class of these embodiments, the glidant is silicon dioxide.

The pharmaceutical compositions of the present invention optionally contain one or more binding agents. Embodiments of binding agents include hydroxypropylcellulose (HPC), hydroxypropylmethyl cellulose (HPMC), hydroxyethyl cellulose, pregelatinized starch, pregelatinized corn starch, starch 1500, corn starch, polyvinylpyrrolidone (povidone), and co-povidone. In one embodiment, the binding agent is polyvinylpyrrolidone. In another embodiment of the present invention, the binding agent is hydroxypropylcellulose. In another embodiment, the binding agent is hydroxypropylcellulose (HPC) in solution. In another embodiment, the binding agent is hydroxypropylcellulose (HPC) in an aqueous solution.

The pharmaceutical compositions of the present invention may also optionally contain one or more diluents. Examples of diluents include mannitol, sorbitol, anhydrous dibasic calcium phosphate, anhydrous lactose, lactose monohydrate, lactose, dibasic calcium phosphate dihydrate, microcrystalline cellulose, and powdered cellulose. Microcrystalline cellulose is available from several suppliers and includes Avicel, Avicel PH 101, Avicel PH 102, Avicel, PH 103, Avicel PH 105, Avicel PH 112, Avicel PH 200, Avicel PH 301, Avicel PH 302 and Avicel PH PH 200LM, manufactured by the FMC Corporation. In one embodiment of the present invention, microcrystalline cellulose is selected from: Avicel, Avicel PH 101, Avicel PH 102, Avicel, PH 103, Avicel PH 105, Avicel PH 112, Avicel PH 200, Avicel PH 301, Avicel PH 302 and Avicel PH 200LM. Microcrystalline cellulose is also available from several suppliers in combination with other ingredients, and includes Prosolv®SMCC (which contains 98% microcrystalline cellulose and 2% silicon dioxide), Prosolv® SMCC 50, Prosolv® SMCC 90, Prosolv® SMCC HD90, and Prosolv® SMCC 90 LM, manufactured by JRS PHARMA GmbH & Co. KG. In one embodiment of the present invention, the microcrystalline cellulose is Avicel PH 102.

In another embodiment of the present invention, microcrystalline cellulose is silicified microcrystalline cellulose which contains silicon dioxide (SiO₂). In another embodiment of the present invention, microcrystalline cellulose is silicified microcrystalline cellulose which contains up to 2% of silicon dioxide (SiO₂). In another embodiment of the present invention, microcrystalline cellulose is Prosolv®. In another embodiment of the present invention, microcrystalline cellulose is Prosolv®SMCC. In another embodiment of the present invention, microcrystalline cellulose is Prosolv® SMCC 50. In another embodiment of the present invention, microcrystalline cellulose is Prosolv® SMCC 90. In another embodiment of the present invention, microcrystalline cellulose is Prosolv® SMCC HD90. In another embodiment of the present invention, microcrystalline cellulose is Prosolv® SMCC 90LM.

In another embodiment of the present invention, the pharmaceutical composition optionally contains a glidant. In a class of this embodiment, the glidant is silicon dioxide. In a subclass of this class, the glidant is silicon dioxide. In a subclass of this class, the silicon dioxide is 2% of the microcrystalline cellulose used in one layer or both layers of the bilayer tablet. In another subclass of this class, the silicon dioxide is 2% of the microcrystalline cellulose used in one layer of the bilayer tablet.

In another embodiment the diluent is selected from: mannitol, anhydrous dibasic calcium phosphate, anhydrous lactose and microcrystalline cellulose, or a mixture of any two, three or four thereof. In another embodiment the diluent is selected from: mannitol, dibasic calcium phosphate, lactose and microcrystalline cellulose, or a mixture of any two, three or four thereof.

In another embodiment the diluent is selected from: anhydrous dibasic calcium phosphate, dibasic calcium phosphate, and microcrystalline cellulose, or a mixture thereof. In another embodiment the diluent is a mixture of anhydrous dibasic calcium phosphate and microcrystalline cellulose.

In another embodiment the diluent is selected from: mannitol, lactose and microcrystalline cellulose, or a mixture of any two or three thereof. In another embodiment the diluent is selected from: mannitol, anhydrous lactose and microcrystalline cellulose, or a mixture of any two or three thereof.

In another embodiment, the diluent is a 2:1 to 1:2 mixture of microcrystalline cellulose to lactose or microcrystalline cellulose to mannitol. In another embodiment, the diluent is a 2:1 to 1:2 mixture of microcrystalline cellulose to anhydrous lactose or microcrystalline cellulose to mannitol. In another embodiment, the diluent is a 1:1 mixture of microcrystalline cellulose to lactose. In another embodiment, the diluent is a 1:1 mixture of microcrystalline cellulose to anhydrous lactose. In another embodiment, the diluent is a 1:1 mixture of microcrystalline cellulose to mannitol. In another embodiment, the diluent is a 1.5:1 mixture of microcrystalline cellulose to lactose or microcrystalline cellulose to mannitol. In another embodiment, the diluent is a 1.5:1 mixture of microcrystalline cellulose to anhydrous lactose or microcrystalline cellulose to mannitol. In another embodiment, the diluent is a 1.5:1 mixture of microcrystalline cellulose to lactose. In another embodiment, the diluent is a 1.5:1 mixture of microcrystalline cellulose to anhydrous lactose. In another embodiment, the diluent is a 1.5:1 mixture of microcrystalline cellulose to mannitol.

In another embodiment the diluent is selected from: lactose and microcrystalline cellulose, or a mixture thereof. In another embodiment the diluent is selected from: anhydrous lactose and microcrystalline cellulose, or a mixture thereof. In another embodiment the diluent is a mixture of lactose and microcrystalline cellulose. In another embodiment the diluent is a mixture of anhydrous lactose and microcrystalline cellulose. In another embodiment, the diluent is a 2:1 to 1:2 mixture of microcrystalline cellulose to lactose. In another embodiment, the diluent is a 2:1 to 1:2 mixture of microcrystalline cellulose to anhydrous lactose. In another embodiment, the diluent is a 1:1 mixture of microcrystalline cellulose to lactose. In another embodiment, the diluent is a 1:1 mixture of microcrystalline cellulose to anhydrous lactose. In another embodiment, the diluent is a 1.5:1 mixture of microcrystalline cellulose to anhydrous lactose.

In another embodiment the diluent is selected from: mannitol and microcrystalline cellulose, or a mixture thereof. In another embodiment the diluent is a mixture of mannitol and microcrystalline cellulose. In another embodiment, the diluent is a mixture of microcrystalline cellulose and mannitol. In another embodiment, the diluent is a 2:1 to 1:2 mixture of microcrystalline cellulose to mannitol. In another embodiment, the diluent is a 1:1 mixture of microcrystalline cellulose to mannitol. In another embodiment, the diluent is a 1.5:1 mixture of microcrystalline cellulose to mannitol.

In another embodiment, the diluent is microcrystalline cellulose. In another embodiment, the diluent is mannitol. In another embodiment of the present invention, the diluent is lactose. In another embodiment of the present invention, the diluent is anhydrous lactose.

In another embodiment the diluent is selected from: mannitol, anhydrous dibasic calcium phosphate, anhydrous lactose, lactose, lactose monohydrate and microcrystalline cellulose, or a mixture of any two, three or four thereof. In another embodiment the diluent is selected from: mannitol, lactose monohydrate and microcrystalline cellulose, or a mixture of any two or three thereof. In another embodiment the diluent is selected from: lactose, lactose monohydrate and microcrystalline cellulose, or a mixture thereof. In another embodiment the diluent is selected from: lactose monohydrate and microcrystalline cellulose, or a mixture thereof. In another embodiment the diluent is a mixture of lactose monohydrate and microcrystalline cellulose.

The pharmaceutical compositions of the present invention may also optionally contain a disintegrant. The disintegrant may be one of several modified starches, modified cellulose polymers, or polycarboxylic acids, such as croscarmellose sodium, sodium starch glycolate, polacrillin potassium, carboxymethylcellulose calcium (CMC Calcium), and crospovidone. In one embodiment, the disintegrant is selected from: polacrillin potassium, croscarmellose sodium, carboxymethylcellulose calcium (CMC Calcium), and crospovidone. In another embodiment, the disintegrant is crospovidone and croscarmellose sodium. In another embodiment, the disintegrant is crospovidone. In another embodiment of the present invention, the disintegrant is croscarmellose sodium.

The pharmaceutical compositions of the present invention may also optionally contain one or more surfactants or wetting agents. The surfactant may be anionic, cationic, or neutral. Anionic surfactants include sodium lauryl sulfate, sodium dodecanesulfonate, sodium oleyl sulfate, and sodium laurate mixed with stearates and talc. Cationic surfactants include benzalkonium chlorides and alkyltrimethylammonium bromides. Neutral and non-ionic surfactants include polysorbate, polysorbate 80, glyceryl monooleate, polyoxyethylene sorbitan fatty acid esters, polyvinyl alcohol, and sorbitan esters. Embodiments of wetting agents include poloxamer, polyoxyethylene alkyl ethers, polyoxyethylene castor oil derivatives, and polyoxyethylene stearates. In one embodiment of the present invention, the surfactant is sodium lauryl sulfate. In another embodiment of the present invention, the surfactant is polysorbate. In another embodiment of the present invention, the surfactant is polysorbate 80.

The pharmaceutical compositions of the present invention may also contain an alkalizing agent. The alkalizing agent includes, but it not limited to, L-arginine, calcium carbonate and sodium bicarbonate. In one embodiment of the present invention, the alkalizing agent is selected from the group consisting of L-arginine, calcium carbonate and sodium bicarbonate. In another embodiment of the present invention, the alkalizing agent is selected from the group consisting of L-arginine and sodium bicarbonate. In another embodiment of the present invention, the alkalizing agent is L-arginine. In another embodiment of the present invention, the alkalizing agent is sodium bicarbonate. In another embodiment of the present invention, the alkalizing agent is calcium carbonate.

The pharmaceutical compositions of the present invention may also optionally contain an anti-oxidant which may be added to the formulation to impart chemical stability. The anti-oxidant is selected from the group consisting of α-tocopherol, γ-tocopherol, δ-tocopherol, extracts of natural origin rich in tocopherol, citric acid, citric acid monohydrate, ascorbic acid, L-ascorbic acid and its sodium or calcium salts, ascorbyl palmitate, propyl gallate, octyl gallate, dodecyl gallate, butylated hydroxytoluene (BHT), and butylated hydroxyanisole (BHA). In one embodiment, the antioxidant is butylated hydroxyanisole. In another embodiment, the antioxidant is citric acid. In another embodiment, the antioxidant is citric acid monohydrate. In another embodiment, the antioxidant is ascorbic acid. In another embodiment of the present invention, the anti-oxidant is a mixture of butylated hydroxyanisole, citric acid or citric acid monohydrate and ascorbic acid. In another embodiment of the present invention, the anti-oxidant is a mixture of butylated hydroxyanisole, citric acid and ascorbic acid. In another embodiment of the present invention, the anti-oxidant is a mixture of butylated hydroxyanisole, citric acid monohydrate and ascorbic acid.

Preferred dosage forms for the pharmaceutical compositions of the present invention are tablets which are prepared by compression methods. Such tablets may be film-coated such as with a mixture of hydroxypropylcellulose and hydroxypropylmethylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; or any other suitable immediate-release film-coating agent(s). The coat provides taste masking and additional stability to the final tablet. A commercial film-coating agent is Opadry® which is a formulated powder blend provided by Colorcon. Embodiments of Opadry® useful in the present invention include, but are not limited to, Opagloss 2, Opagloss II, Opadry® I (HPC/HPMC), Opadry® 20A18334, Opadry® II, Opadry® II HP (PVA-PEG), Purple Opadry® [85F170000], Beige Opadry® [85F170001], Opadry® Red, Opadry® Red-Orange, or another suitable Opadry® suspension (such as polyvinyl alcohol, polyethylene glycol, titanium dioxide, and talc, with or without colorants).

Compositions of the present invention are optionally and preferably stored in protective packaging to minimize or prevent degradation of the composition due to exposure to oxygen and/or water. When present, suitable packaging includes desiccants, oxygen scavengers, anti-oxidants, vacuum packing, nitrogen header space and the like. For example, bilayer tablets of the present invention containing amorphous atorvastatin calcium salt can be stored in a container such as a sealed bottle or foil pouch which contains an oxygen scavenger with or without a desiccant to reduce degradation during storage.

Finally, a sweetening agent and/or flavoring agent may be added if desired.

In one embodiment of the present invention, the pharmaceutical composition comprises:

(a) a first layer comprising about 20 to 45% by weight of a dipeptidyl peptidase-4 inhibitor, or a pharmaceutically acceptable salt thereof; and
(b) a second layer comprising about 5 to 15% by weight of atorvastatin, or a pharmaceutically acceptable salt thereof.

In a class of this embodiment, the first layer additionally comprises one or more excipients selected from the group consisting of: (i) a diluent; (ii) a disintegrant; and (iii) a lubricant. In a subclass of this class, the first layer additionally comprises one or more excipients selected from the group consisting of: (i) two diluents; (ii) a disintegrant; and (iii) two lubricants.

In another class of this embodiment, the first layer additionally comprises one or more excipients selected from the group consisting of (i) about 40-80% by weight of a diluent; (ii) about 0.1-10% by weight of a disintegrant; and (iii) about 0.5-10% by weight of a lubricant. In a subclass of this class, the first layer additionally comprises one or more excipients selected from the group consisting of: (i) about 40-80% by weight of two diluents; (ii) about 0.1-10% by weight of a disintegrant; and (iii) about 0.5-10% by weight of two lubricants.

In another class of this embodiment, the first layer additionally comprises one or more excipients selected from the group consisting of: (i) about 10-60% by weight of a first diluent; (ii) about 10-60% of a second diluent; (iii) about 0.5-10% by weight of a disintegrant; (iv) about 0.25-5% by weight of a first lubricant and (v) about 0.25-5% by weight of a second lubricant. In a subclass of this class, the first diluent is dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the second layer additionally comprises one or more excipients selected from the group consisting of: (i) a diluent; (ii) a binding agent; (iii) a disintegrant; (iv) a surfactant, and (v) a lubricant. In another class of this embodiment, the second layer additionally comprises one or more excipients selected from the group consisting of: (i) two diluents; (ii) one binding agent; (iii) one disintegrant; (iv) one surfactant, and (v) one lubricant.

In another class of this embodiment, the second layer additionally comprises one or more excipients selected from the group consisting of: (i) about 2 to 90% by weight of a diluent; (ii) about 0 to 15% of a binding agent; (iii) about 0.1 to 20% by weight of a disintegrant; (iv) about 0 to 5% by weight of a surfactant, and (v) about 0.25 to 5% by weight of a lubricant. In a subclass of this embodiment, the second layer additionally comprises one or more excipients selected from the group consisting of: (i) about 2 to 90% by weight of two diluents; (ii) about 0 to 15% of a binding agent; (iii) about 0.1 to 20% by weight of a disintegrant; (iv) about 0 to 5% by weight of a surfactant, and (v) about 0.25 to 5% by weight of a lubricant. In another subclass of this class of this embodiment, the second layer additionally comprises one or more excipients selected from the group consisting of (i) about 2 to 60% by weight of a first diluent and about 10 to 90% of a second diluent; (ii) about 0 to 15% of a binding agent; (iii) about 1 to 20% by weight of a disintegrant; (iv) about 0 to 5% by weight of a surfactant, and (v) about 0.25 to 5% by weight of a lubricant. In another subclass of this class of this embodiment, the second layer additionally comprises one or more excipients selected from the group consisting of: (i) about 30 to 40% by weight of a first diluent and about 30 to 40% of a second diluent; (ii) about 0 to 15% of a binding agent; (iii) about 1 to 20% by weight of a disintegrant; (iv) about 0 to 5% by weight of a surfactant, and (v) about 0.25 to 5% by weight of a lubricant.

In another subclass of this class, the diluent in the second layer is selected from the group consisting of: microcrystalline cellulose, anhydrous lactose and mannitol, or a mixture thereof; the binding agent is hydroxypropyl cellulose; the disintegrant is croscarmellose sodium; the surfactant is sodium lauryl sulfate; and the lubricant is selected from the group consisting of: magnesium stearate, and sodium stearyl fumarate, or a mixture thereof. In another subclass of this class, the diluent in the second layer is selected from the group consisting of microcrystalline cellulose, anhydrous lactose and mannitol, or a mixture thereof; the binding agent is hydroxypropyl cellulose; the disintegrant is croscarmellose sodium; the surfactant is sodium lauryl sulfate; and the lubricant is magnesium stearate. In another subclass of this class, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another subclass of this class, the diluent in the second layer is a 1:1 mixture of microcrystalline cellulose and mannitol, or a 1:1 mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate.

In another class of this embodiment, the second layer additionally comprises one or more excipients selected from the group consisting of: (i) a diluent; (ii) one binding agent; (iii) one disintegrant; (iv) one surfactant, and (v) one lubricant.

In another class of this embodiment, the second layer additionally comprises excipients selected from the group consisting of: (i) about 10 to 90% by weight of a diluent; (ii) about 0 to 15% of a binding agent; (iii) about 2 to 20% by weight of a disintegrant; (iv) about 0 to 5% by weight of a surfactant, and (v) about 0.1 to 5% by weight of a lubricant.

In another class of this embodiment, the second layer additionally comprises excipients selected from the group consisting of: (i) about 10 to 90% by weight of two diluents; (ii) about 0 to 15% of a binding agent; (iii) about 2 to 20% by weight of a disintegrant; (iv) about 0 to 5% by weight of a surfactant, and (v) about 0.1 to 5% by weight of a lubricant.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; and the lubricant is selected from the group consisting of: magnesium stearate, and sodium stearyl fumarate, or a mixture thereof. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; and the lubricant is magnesium stearate. In a subclass of this subclass, the second layer further comprises a glidant. In a subclass of this subclass, the second layer further comprises a glidant, wherein the glidant is silicon dioxide.

In another class of this embodiment, the second layer additionally comprises one or more excipients selected from the group consisting of: (i) a diluent; (ii) one binding agent; (iii) one disintegrant; (iv) one surfactant, (v) one lubricant, and (vi) one alkalizing agent.

In another class of this embodiment, the second layer additionally comprises excipients selected from the group consisting of: (i) about 10 to 90% by weight of a diluent; (ii) about 0 to 15% of a binding agent; (iii) about 2 to 20% by weight of a disintegrant; (iv) about 0 to 5% by weight of a surfactant, (v) about 0.1 to 5% by weight of a lubricant, and (vi) about 2 to 50% by weight of an alkalizing agent.

In another class of this embodiment, the second layer additionally comprises excipients selected from the group consisting of: (i) about 10 to 90% by weight of two diluents; (ii) about 0 to 15% of a binding agent; (iii) about 2 to 20% by weight of a disintegrant; (iv) about 0 to 5% by weight of a surfactant, (v) about 0.1 to 5% by weight of a lubricant, and (vi) about 2 to 50% by weight of an alkalizing agent.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of magnesium stearate, and sodium stearyl fumarate, or a mixture thereof; and the alkalizing agent is calcium carbonate. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. In a subclass of this subclass, the second layer further comprises a glidant. In a subclass of this subclass, the second layer further comprises a glidant, wherein the glidant is silicon dioxide.

In another class of this embodiment, the second layer additionally comprises one or more excipients selected from the group consisting of (i) a diluent; (ii) one binding agent; (iii) one disintegrant; (iv) one surfactant, (v) one lubricant, (vi) one alkalizing agent; and (vii) one glidant.

In another class of this embodiment, the second layer additionally comprises excipients selected from the group consisting of: (i) about 10 to 90% by weight of a diluent; (ii) about 0 to 15% of a binding agent; (iii) about 2 to 20% by weight of a disintegrant; (iv) about 0 to 5% by weight of a surfactant, (v) about 0.1 to 5% by weight of a lubricant, (vi) about 2 to 50% by weight of an alkalizing agent, and (vii) about 0.1 to 5% by weight of a glidant.

In another class of this embodiment, the second layer additionally comprises excipients selected from the group consisting of: (i) about 10 to 90% by weight of two diluents; (ii) about 0 to 15% of a binding agent; (iii) about 2 to 20% by weight of a disintegrant; (iv) about 0 to 5% by weight of a surfactant, (v) about 0.1 to 5% by weight of a lubricant, (vi) about 2 to 50% by weight of an alkalizing agent, and (vii) about 0.1 to 5% by weight of a glidant.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of: magnesium stearate, and sodium stearyl fumarate, or a mixture thereof; the alkalizing agent is calcium carbonate; and the glidant is silicon dioxide.

In another subclass of this class, the diluent in the second layer is a mixture of microcrystalline cellulose and lactose monohydrate; the binding agent is hydroxypropyl cellulose; the disintegrant is croscannellose sodium; the surfactant is polysorbate 80; the lubricant is magnesium stearate; the alkalizing agent is calcium carbonate; and the glidant is silicon dioxide.

In another class of this embodiment, the second layer of the pharmaceutical composition (the atorvastatin layer) contains about 0 to 4% of an alkalizing agent. In a subclass of this class, the composition contains about 0.2 to 4% of an alkalizing agent. In another subclass of this class, the composition contains about 4% of an alkalizing agent. In another subclass of this class, the alkalizing agent is selected from the group consisting of L-arginine, calcium carbonate and sodium bicarbonate. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of this embodiment, the second layer of the pharmaceutical composition (the atorvastatin layer) contains about 2 to 50% of an alkalizing agent. In a subclass of this class, the composition contains about 22 to 40% of an alkalizing agent. In another subclass of this class, the composition contains about 29 to 33% of an alkalizing agent. In another subclass of this class, the composition contains about 31% of an alkalizing agent. In another subclass of this class, the alkalizing agent is selected from the group consisting of L-arginine, calcium carbonate and sodium bicarbonate. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of this embodiment, the dipeptidyl peptidase-4 inhibitor is selected from the group consisting of: alogliptin, carmegiptin, denagliptin, dutogliptin, linagliplin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another embodiment of the present invention, atorvastatin is atorvastatin free acid. In another embodiment of the present invention, atorvastatin is selected from the group consisting of: atorvastatin, or a pharmaceutically acceptable salt, hydrate and solvate thereof. In another embodiment of the present invention, atorvastatin is selected from the group consisting of: atorvastatin, or a pharmaceutically acceptable salt and hydrate thereof. In another embodiment of the present invention, atorvastatin is selected from the group consisting of atorvastatin calcium, or a hydrate thereof. In another embodiment of the present invention, atorvastatin is selected from the group consisting of: atorvastatin calcium salt (2:1), or a hydrate thereof. In another embodiment of the present invention, atorvastatin is selected from the group consisting of: atorvastatin calcium, or a trihydrate thereof. In another embodiment of the present invention, atorvastatin is selected from the group consisting of: atorvastatin calcium salt (2:1), or a trihydrate thereof. In a class of the embodiment of the present invention, atorvastatin calcium is amorphous. In another class of the embodiment of the present invention, atorvastatin calcium is crystalline. In another class of the embodiment of the present invention, atorvastatin calcium salt (2:1) is amorphous. In another class of the embodiment of the present invention, atorvastatin calcium salt (2:1) is crystalline. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

In a second embodiment of the present invention, the pharmaceutical composition comprises:

(a) a first layer comprising:

(i) about 20 to 45% by weight of a dipeptidyl peptidase-4 inhibitor, or a pharmaceutically acceptable salt thereof;

(ii) about 40 to 80% by weight of a diluent;

(iii) about 0.5 to 10% by weight of a disintegrant; and

(iv) about 0.5 to 10% by weight of a lubricant; and

(b) a second layer comprising:

(i) about 5 to 15% by weight of atorvastatin;

(ii) about 60 to 80% by weight of a diluent;

(iii) about 0 to 5% of a binding agent;

(iv) about 1 to 10% by weight of a disintegrant;

(v) about 0 to 3% by weight of a surfactant, and

(vi) about 0.5 to 3% by weight of a lubricant.

In another class of this embodiment, the first layer additionally comprises one or more excipients selected from the group consisting of: (i) about 40-80% by weight of two diluents; (ii) about 0.5 to 10% by weight of a disintegrant; and (iii) about 0.5 to 10% by weight of a lubricant. In another class of this embodiment, the first layer additionally comprises one or more excipients selected from the group consisting of: (i) about 10-60% by weight of a first diluent; (ii) about 10-60% of a second diluent; (iii) about 0.5-10% by weight of a disintegrant; (iv) about 0.25-5% by weight of a first lubricant and (v) about 0.25-5% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the pharmaceutical composition comprises a first layer wherein the diluent is selected from the group consisting of: microcrystalline cellulose, mannitol and anhydrous dibasic calcium phosphate, or a mixture thereof; the disintegrant is selected from the group consisting of crospovidone and croscarmellose sodium, or a mixture thereof; and the lubricant is selected from the group consisting of: magnesium stearate and sodium stearyl fumarate, or a mixture thereof. In another class of this embodiment, the pharmaceutical composition comprises a first layer wherein the diluent is a mixture of microcrystalline cellulose and anhydrous dibasic calcium phosphate; the disintegrant is croscarmellose sodium; and the lubricant is a mixture of magnesium stearate and sodium stearyl fumarate.

In another class of this embodiment, the second layer additionally comprises one or more excipients selected from the group consisting of: (i) about 60-80% by weight of two diluents; (ii) about 0 to 5% of a binding agent; (iii) about 1 to 10% by weight of a disintegrant; (iv) about 0 to 3% by weight of a surfactant, and (v) about 0.5 to 4.5% by weight of a lubricant. In another class of this embodiment, the first layer additionally comprises one or more excipients selected from the group consisting of: (i) about 2-60% by weight of a first diluent; (ii) about 10-90% of a second diluent; (iii) about 0 to 5% of a binding agent; (iv) about 2 to 10% by weight of a disintegrant; (v) about 0 to 3% by weight of a surfactant, and (vi) about 0.5 to 3% by weight of a lubricant.

In a subclass of this class, the diluent in the second layer is selected from the group consisting of: microcrystalline cellulose, anhydrous lactose and mannitol, or a mixture thereof; the binding agent is hydroxypropyl cellulose; the disintegrant is croscarmellose sodium; the surfactant is sodium lauryl sulfate; and the lubricant is selected from the group consisting of magnesium stearate, and sodium stearyl fumarate, or a mixture thereof. In another subclass of this class, the diluent in the second layer is selected from the group consisting of: microcrystalline cellulose, anhydrous lactose and mannitol, or a mixture thereof; the binding agent is hydroxypropyl cellulose; the disintegrant is croscarmellose sodium; the surfactant is sodium lauryl sulfate; and the lubricant is magnesium stearate. In another subclass of this class, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another subclass of this class, the diluent in the second layer is a 1:1 mixture of microcrystalline cellulose and mannitol, or a 1:1 mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate.

In another class of this embodiment, the second layer of the pharmaceutical composition (the atorvastatin layer) contains about 0 to 4% of an alkalizing agent. In a subclass of this class, the composition contains about 0.2 to 4% of an alkalizing agent. In another subclass of this class, the composition contains about 4% of an alkalizing agent. In another subclass of this class, the alkalizing agent is selected from the group consisting of L-arginine, calcium carbonate and sodium bicarbonate. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another embodiment of the present invention, atorvastatin is atorvastatin free acid. In another embodiment of the present invention, atorvastatin is selected from the group consisting of: atorvastatin, or a pharmaceutically acceptable salt, hydrate and solvate thereof. In another embodiment of the present invention, atorvastatin is selected from the group consisting of atorvastatin, or a pharmaceutically acceptable salt and hydrate thereof. In another embodiment of the present invention, atorvastatin is selected from the group consisting of: atorvastatin calcium, or a hydrate thereof. In another embodiment of the present invention, atorvastatin is selected from the group consisting of: atorvastatin calcium salt (2:1), or a hydrate thereof. In another embodiment of the present invention, atorvastatin is selected from the group consisting of: atorvastatin calcium, or a trihydrate thereof. In another embodiment of the present invention, atorvastatin is selected from the group consisting of: atorvastatin calcium salt (2:1), or a trihydrate thereof. In a class of the embodiment of the present invention, atorvastatin calcium is amorphous. In another class of the embodiment of the present invention, atorvastatin calcium is crystalline. In another class of the embodiment of the present invention, atorvastatin calcium salt (2:1) is amorphous. In another class of the embodiment of the present invention, atorvastatin calcium salt (2:1) is crystalline. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

In a third embodiment of the present invention, the pharmaceutical composition comprises:

(a) a first layer comprising:

(i) about 25 to 35% by weight of a dipeptidyl peptidase-4 inhibitor, or a pharmaceutically acceptable salt thereof;

(ii) about 50-70% by weight of a diluent;

(iii) about 1-4% by weight of a disintegrant; and

(iv) about 0.5-10% by weight of a lubricant; and

(b) a second layer comprising:

(i) about 5 to 15% by weight of atorvastatin;

(ii) about 60 to 80% by weight of a diluent;

(iii) about 0 to 5% of a binding agent;

(iv) about 1 to 10% by weight of a disintegrant;

(v) about 0 to 3% by weight of a surfactant, and

(vi) about 0.5 to 3% by weight of a lubricant.

In a class of this embodiment, the pharmaceutical composition comprises a first layer comprising: (i) about 25 to 35% by weight of a dipeptidyl peptidase-4 inhibitor, or a pharmaceutically acceptable salt thereof; (ii) about 50-70% by weight of two diluents; (iii) about 1-4% by weight of a disintegrant; and (iv) about 1.5-10% by weight of two lubricants.

In another class of this embodiment, the first layer additionally comprises one or more excipients selected from the group consisting of (i) about 20-40% by weight of a first diluent (ii) about 20-40% of a second diluent; (iii) about 1-4% by weight of a disintegrant; (iv) about 0.25-5% by weight of a first lubricant and (v) about 0.25-5% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the pharmaceutical composition comprises a first layer wherein the dipeptidyl peptidase-4 inhibitor is sitagliptin, or a pharmaceutically acceptable salt thereof; the diluent is a mixture of microcrystalline cellulose and anhydrous dibasic calcium phosphate; the disintegrant is croscarmellose sodium; and the lubricant is a mixture of magnesium stearate and sodium stearyl fumarate.

In a class of this embodiment, the pharmaceutical composition comprises a second layer comprising: (i) about 5 to 15% by weight of atorvastatin; (ii) about 60 to 80% by weight of a diluent; (iii) about 0 to 5% of a binding agent; (iv) about 1 to 10% by weight of a disintegrant; (v) about 0 to 3% by weight of a surfactant, and (vi) about 0.5 to 3% by weight of a lubricant.

In another class of this embodiment, the pharmaceutical composition comprises a second layer comprising: (i) about 5 to 15% by weight of atorvastatin; (ii) about 20-40% by weight of the first diluent; (iii) about 20-40% by weight of the second diluent; (iv) about 0 to 5% of a binding agent; (v) about 1 to 10% by weight of a disintegrant; (vi) about 0 to 3% by weight of a surfactant, and (vii) about 0.5 to 3% by weight of a lubricant.

In another subclass of this class, the diluent in the second layer is selected from the group consisting of: microcrystalline cellulose, anhydrous lactose and mannitol, or a mixture thereof; the binding agent is hydroxypropyl cellulose; the disintegrant is croscarmellose sodium; the surfactant is sodium lauryl sulfate; and the lubricant is selected from the group consisting of: magnesium stearate, and sodium stearyl fumarate, or a mixture thereof. In another subclass of this class, the diluent in the second layer is selected from the group consisting of: microcrystalline cellulose, anhydrous lactose and mannitol, or a mixture thereof; the binding agent is hydroxypropyl cellulose; the disintegrant is croscarmellose sodium; the surfactant is sodium lauryl sulfate; and the lubricant is magnesium stearate. In another subclass of this class, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another subclass of this class, the diluent in the second layer is a 1:1 mixture of microcrystalline cellulose and mannitol, or a 1:1 mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate.

In another class of this embodiment, the second layer of the pharmaceutical composition (the atorvastatin layer) contains about 0 to 4% of an alkalizing agent. In a subclass of this class, the composition contains about 0.2 to 4% of an alkalizing agent. In another subclass of this class, the composition contains about 4% of an alkalizing agent. In another subclass of this class, the alkalizing agent is selected from the group consisting of L-arginine, calcium carbonate and sodium bicarbonate. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another embodiment of the present invention, atorvastatin is atorvastatin free acid. In another embodiment of the present invention, atorvastatin is selected from the group consisting of: atorvastatin, or a pharmaceutically acceptable salt, hydrate and solvate thereof. In another embodiment of the present invention, atorvastatin is selected from the group consisting of: atorvastatin, or a pharmaceutically acceptable salt and hydrate thereof. In another embodiment of the present invention, atorvastatin is selected from the group consisting of: atorvastatin calcium, or a hydrate thereof. In another embodiment of the present invention, atorvastatin is selected from the group consisting of atorvastatin calcium salt (2:1), or a hydrate thereof.

In another embodiment of the present invention, atorvastatin is selected from the group consisting of: atorvastatin calcium, or a trihydrate thereof. In another embodiment of the present invention, atorvastatin is selected from the group consisting of: atorvastatin calcium salt (2:1), or a trihydrate thereof. In a class of the embodiment of the present invention, atorvastatin calcium is amorphous. In another class of the embodiment of the present invention, atorvastatin calcium is crystalline. In another class of the embodiment of the present invention, atorvastatin calcium salt (2:1) is amorphous. In another class of the embodiment of the present invention, atorvastatin calcium salt (2:1) is crystalline. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

In a fourth embodiment of the present invention, the pharmaceutical composition comprises:

(a) a first layer comprising:

(i) about 30 to 35% by weight of a dipeptidyl peptidase-4 inhibitor, or a pharmaceutically acceptable salt thereof;

(ii) about 50-70% by weight of a diluent;

(iii) about 1-4% by weight of a disintegrant; and

(iv) about 0.5-7% by weight of a lubricant; and

(b) a second layer comprising:

(i) about 9 to 11% by weight of atorvastatin;

(ii) about 72 to 76% by weight of a diluent;

(iii) about 2 to 4% of a binding agent;

(iv) about 5 to 7% by weight of a disintegrant;

(v) about 0 to 2% by weight of a surfactant, and

(vi) about 1 to 2% by weight of a lubricant.

In a class of this embodiment, the pharmaceutical composition comprises a first layer comprising: (i) about 30 to 35% by weight of a dipeptidyl peptidase-4 inhibitor, or a pharmaceutically acceptable salt thereof; (ii) about 50-70% by weight of two diluents; (iii) about 1-4% by weight of a disintegrant; and (iv) about 0.5-7% by weight of two lubricants.

In another class of this embodiment, the first layer additionally comprises one or more excipients selected from the group consisting of: (i) about 20-40% by weight of a first diluent; (ii) about 20-40% of a second diluent; (iii) about 1-4% by weight of a disintegrant; (iv) about 0.25-5% by weight of a first lubricant and (v) about 0.25-5% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the pharmaceutical composition comprises a first layer wherein the dipeptidyl peptidase-4 inhibitor is sitagliptin, or a pharmaceutically acceptable salt thereof; the diluent is a mixture of microcrystalline cellulose and anhydrous dibasic calcium phosphate; the disintegrant is croscarmellose sodium; and the lubricant is a mixture of magnesium stearate and sodium stearyl fumarate.

In a class of this embodiment, the pharmaceutical composition comprises a second layer comprising: (i) about 9 to 11% by weight of atorvastatin; (ii) about 72 to 76% by weight of two diluents; (iii) about 2 to 4% of a binding agent; (iv) about 5 to 7% by weight of a disintegrant; (v) about 0 to 2% by weight of a surfactant, and (vi) about 1 to 2% by weight of a lubricant.

In another class of this embodiment, the pharmaceutical composition comprises a second layer comprising: (i) about 9 to 11% by weight of a atorvastatin; (ii) about 36-38% by weight of the first diluent; (iii) about 36-38% by weight of the second diluent; (iv) about 2 to 4% of a binding agent; (v) about 5 to 7% by weight of a disintegrant; (vi) about 0 to 2% by weight of a surfactant, and (vii) about 1 to 2% by weight of a lubricant.

In another subclass of this class, the diluent in the second layer is selected from the group consisting of: microcrystalline cellulose, anhydrous lactose and mannitol, or a mixture thereof; the binding agent is hydroxypropyl cellulose; the disintegrant is croscarmellose sodium; the surfactant is sodium lauryl sulfate; and the lubricant is selected from the group consisting of: magnesium stearate, and sodium stearyl fumarate, or a mixture thereof. In another subclass of this class, the diluent in the second layer is selected from the group consisting of: microcrystalline cellulose, anhydrous lactose and mannitol, or a mixture thereof; the binding agent is hydroxypropyl cellulose; the disintegrant is croscarmellose sodium; the surfactant is sodium lauryl sulfate; and the lubricant is magnesium stearate. In another subclass of this class, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate, and the lubricant is magnesium stearate. In another subclass of this class, the diluent in the second layer is a 1:1 mixture of microcrystalline cellulose and mannitol, or a 1:1 mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate.

In another class of this embodiment, the second layer of the pharmaceutical composition (the atorvastatin layer) contains about 0 to 4% of an alkalizing agent. In a subclass of this class, the composition contains about 0.2 to 4% of an alkalizing agent. In another subclass of this class, the composition contains about 4% of an alkalizing agent. In another subclass of this class, the alkalizing agent is selected from the group consisting of L-arginine, calcium carbonate and sodium bicarbonate. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another embodiment of the present invention, atorvastatin is atorvastatin free acid. In another embodiment of the present invention, atorvastatin is selected from the group consisting of: atorvastatin, or a pharmaceutically acceptable salt, hydrate and solvate thereof. In another embodiment of the present invention, atorvastatin is selected from the group consisting of: atorvastatin, or a pharmaceutically acceptable salt and hydrate thereof. In another embodiment of the present invention, atorvastatin is selected from the group consisting of: atorvastatin calcium, or a hydrate thereof. In another embodiment of the present invention, atorvastatin is selected from the group consisting of: atorvastatin calcium salt (2:1), or a hydrate thereof. In another embodiment of the present invention, atorvastatin is selected from the group consisting of: atorvastatin calcium, or a trihydrate thereof. In another embodiment of the present invention, atorvastatin is selected from the group consisting of: atorvastatin calcium salt (2:1), or a trihydrate thereof. In a class of the embodiment of the present invention, atorvastatin calcium is amorphous. In another class of the embodiment of the present invention, atorvastatin calcium is crystalline. In another class of the embodiment of the present invention, atorvastatin calcium salt (2:1) is amorphous. In another class of the embodiment of the present invention, atorvastatin calcium salt (2:1) is crystalline. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

In a fifth embodiment of the present invention, the pharmaceutical composition comprises:

(a) a first layer comprising:

(i) about 20 to 45% by weight of a dipeptidyl peptidase-4 inhibitor, or a pharmaceutically acceptable salt thereof;

(ii) about 40 to 80% by weight of a diluent;

(iii) about 0.5 to 10% by weight of a disintegrant; and

(iv) about 0.5 to 10% by weight of a lubricant; and

(b) a second layer comprising:

(i) about 5 to 15% by weight of atorvastatin;

(ii) about 10 to 90% by weight of a diluent;

(iii) about 0 to 15% of a binding agent;

(iv) about 2 to 20% by weight of a disintegrant;

(v) about 0 to 5% by weight of a surfactant, and

(vi) about 0.1 to 5% by weight of a lubricant.

In another class of this embodiment, the first layer additionally comprises one or more excipients selected from the group consisting of: (i) about 40-80% by weight of two diluents; (ii) about 0.5 to 10% by weight of a disintegrant; and (iii) about 0.5 to 10% by weight of a lubricant. In another class of this embodiment, the first layer additionally comprises one or more excipients selected from the group consisting of: (i) about 10-60% by weight of a first diluent; (ii) about 10-60% of a second diluent; (iii) about 0.5-10% by weight of a disintegrant; (iv) about 0.25-5% by weight of a first lubricant and (v) about 0.25-5% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the pharmaceutical composition comprises a first layer wherein the diluent is selected from the group consisting of: microcrystalline cellulose, mannitol and anhydrous dibasic calcium phosphate, or a mixture thereof; the disintegrant is selected from the group consisting of: crospovidone and croscarmellose sodium, or a mixture thereof; and the lubricant is selected from the group consisting of: magnesium stearate and sodium stearyl fumarate, or a mixture thereof. In another class of this embodiment, the pharmaceutical composition comprises a first layer wherein the diluent is a mixture of microcrystalline cellulose and anhydrous dibasic calcium phosphate; the disintegrant is croscarmellose sodium; and the lubricant is a mixture of magnesium stearate and sodium stearyl fumarate.

In a class of this embodiment, the pharmaceutical composition comprises a second layer comprising: (i) about 5 to 15% by weight of atorvastatin; (ii) about 10 to 90% by weight of two diluents; (iii) about 0 to 15% of a binding agent; (iv) about 2 to 20% by weight of a disintegrant; (v) about 0 to 5% by weight of a surfactant, and (vi) about 0.1 to 5% by weight of a lubricant.

In another class of this embodiment, the pharmaceutical composition comprises a second layer comprising: (i) about 5 to 15% by weight of a atorvastatin; (ii) about 2 to 60% by weight of the first diluent; (iii) about 5 to 90% by weight of the second diluent; (iv) about 0 to 15% of a binding agent; (v) about 2 to 20% by weight of a disintegrant; (vi) about 0 to 5% by weight of a surfactant, and (vii) about 0.1 to 5% by weight of a lubricant.

In another subclass of this class the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; and the lubricant is selected from the group consisting of: magnesium stearate, and sodium stearyl fumarate, or a mixture thereof. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; and the lubricant is magnesium stearate. In a subclass of this subclass, the second layer further comprises a glidant. In a subclass of this subclass, the second layer further comprises a glidant, wherein the glidant is silicon dioxide.

In a class of this embodiment, the pharmaceutical composition comprises a second layer comprising: (i) about 5 to 15% by weight of atorvastatin; (ii) about 10 to 90% by weight of two diluents; (iii) about 0 to 15% of a binding agent; (iv) about 2 to 20% by weight of a disintegrant; (v) about 0 to 5% by weight of a surfactant, (vi) about 0.1 to 5% by weight of a lubricant; and (vi) about 2 to 50% by weight of an alkalizing agent.

In another class of this embodiment, the pharmaceutical composition comprises a second layer comprising: (i) about 5 to 15% by weight of a atorvastatin; (ii) about 2 to 60% by weight of the first diluent; (iii) about 5 to 90% by weight of the second diluent; (iv) about 0 to 15% of a binding agent; (v) about 2 to 20% by weight of a disintegrant; (vi) about 0 to 5% by weight of a surfactant, (vii) about 0.1 to 5% by weight of a lubricant; and (vi) about 2 to 50% by weight of an alkalizing agent.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of: magnesium stearate, and sodium stearyl fumarate, or a mixture thereof; and the alkalizing agent is calcium carbonate. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. In a subclass of this subclass, the second layer further comprises a glidant. In a subclass of this subclass, the second layer further comprises a glidant, wherein the glidant is silicon dioxide.

In a class of this embodiment, the pharmaceutical composition comprises a second layer comprising: (i) about 5 to 15% by weight of atorvastatin; (ii) about 10 to 90% by weight of two diluents; (iii) about 0 to 15% of a binding agent; (iv) about 2 to 20% by weight of a disintegrant; (v) about 0 to 5% by weight of a surfactant, (vi) about 0.1 to 5% by weight of a lubricant; (vi) about 2 to 50% by weight of an alkalizing agent, and (vii) about 0 to 5% by weight of a glidant.

In another class of this embodiment, the pharmaceutical composition comprises a second layer comprising: (i) about 5 to 15% by weight of a atorvastatin; (ii) about 2 to 60% by weight of the first diluent; (iii) about 5 to 90% by weight of the second diluent; (iv) about 0 to 15% of a binding agent; (v) about 2 to 20% by weight of a disintegrant; (vi) about 0 to 5% by weight of a surfactant, (vii) about 0.1 to 5% by weight of a lubricant; (vi) about 2 to 50% by weight of an alkalizing agent, and (vii) about 0 to 5% by weight of a glidant.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of: magnesium stearate, and sodium stearyl fumarate, or a mixture thereof; the alkalizing agent is calcium carbonate; and the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; the alkalizing agent is calcium carbonate; and the glidant is silicon dioxide.

In another class of this embodiment, the second layer of the pharmaceutical composition (the atorvastatin layer) contains about 2 to 50% of an alkalizing agent. In a subclass of this class, the composition contains about 22 to 40% of an alkalizing agent. In another subclass of this class, the composition contains about 29 to 33% of an alkalizing agent. In another subclass of this class, the composition contains about 31% of an alkalizing agent. In another subclass of this class, the alkalizing agent is selected from the group consisting of L-arginine, calcium carbonate and sodium bicarbonate. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another embodiment of the present invention, atorvastatin is atorvastatin free acid. In another embodiment of the present invention, atorvastatin is selected from the group consisting of: atorvastatin, or a pharmaceutically acceptable salt, hydrate and solvate thereof. In another embodiment of the present invention, atorvastatin is selected from the group consisting of atorvastatin, or a pharmaceutically acceptable salt and hydrate thereof. In another embodiment of the present invention, atorvastatin is selected from the group consisting of: atorvastatin calcium, or a hydrate thereof. In another embodiment of the present invention, atorvastatin is selected from the group consisting of: atorvastatin calcium salt (2:1), or a hydrate thereof. In another embodiment of the present invention, atorvastatin is selected from the group consisting of: atorvastatin calcium, or a trihydrate thereof. In another embodiment of the present invention, atorvastatin is selected from the group consisting of: atorvastatin calcium salt (2:1), or a trihydrate thereof. In a class of the embodiment of the present invention, atorvastatin calcium is amorphous. In another class of the embodiment of the present invention, atorvastatin calcium is crystalline. In another class of the embodiment of the present invention, atorvastatin calcium salt (2:1) is amorphous. In another class of the embodiment of the present invention, atorvastatin calcium salt (2:1) is crystalline. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

In a sixth embodiment of the present invention, the pharmaceutical composition comprises:

(a) a first layer comprising:

(i) about 20 to 45% by weight of a dipeptidyl peptidase-4 inhibitor, or a pharmaceutically acceptable salt thereof;

(ii) about 40 to 80% by weight of a diluent;

(iii) about 0.5 to 10% by weight of a disintegrant; and

(iv) about 0.5 to 10% by weight of a lubricant; and

(b) a second layer comprising:

(i) about 5 to 15% by weight of atorvastatin;

(ii) about 30 to 70% by weight of a diluent;

(iii) about 0.1 to 15% of a binding agent;

(iv) about 2 to 20% by weight of a disintegrant;

(v) about 0.1 to 5% by weight of a surfactant, and

(vi) about 0.1 to 5% by weight of a lubricant.

In another class of this embodiment, the first layer additionally comprises one or more excipients selected from the group consisting of: (i) about 40-80% by weight of two diluents; (ii) about 0.5 to 10% by weight of a disintegrant; and (iii) about 0.5 to 10% by weight of a lubricant. In another class of this embodiment, the first layer additionally comprises one or more excipients selected from the group consisting of: (i) about 10-60% by weight of a first diluent; (ii) about 10-60% of a second diluent; (iii) about 0.5-10% by weight of a disintegrant; (iv) about 0.25-5% by weight of a first lubricant and (v) about 0.25-5% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the pharmaceutical composition comprises a first layer wherein the diluent is selected from the group consisting of: microcrystalline cellulose, mannitol and anhydrous dibasic calcium phosphate, or a mixture thereof; the disintegrant is selected from the group consisting of: crospovidone and croscarmellose sodium, or a mixture thereof; and the lubricant is selected from the group consisting of: magnesium stearate and sodium stearyl fumarate, or a mixture thereof. In another class of this embodiment, the pharmaceutical composition comprises a first layer wherein the diluent is a mixture of microcrystalline cellulose and anhydrous dibasic calcium phosphate; the disintegrant is croscarmellose sodium; and the lubricant is a mixture of magnesium stearate and sodium stearyl fumarate.

In a class of this embodiment, the pharmaceutical composition comprises a second layer comprising: (i) about 5 to 15% by weight of atorvastatin; (ii) about 30 to 70% by weight of two diluents; (iii) about 0.1 to 15% of a binding agent; (iv) about 2 to 20% by weight of a disintegrant; (v) about 0.1 to 5% by weight of a surfactant, and (vi) about 0.1 to 5% by weight of a lubricant.

In another class of this embodiment, the pharmaceutical composition comprises a second layer comprising: (i) about 5 to 15% by weight of a atorvastatin; (ii) about 15 to 40% by weight of the first diluent; (iii) about 15 to 40% by weight of the second diluent; (iv) about 0.1 to 15% of a binding agent; (v) about 2 to 20% by weight of a disintegrant; (vi) about 0.1 to 5% by weight of a surfactant, and (vii) about 0.1 to 5% by weight of a lubricant.

In another subclass of this class the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; and the lubricant is selected from the group consisting of: magnesium stearate, and sodium stearyl fumarate, or a mixture thereof. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; and the lubricant is magnesium stearate. In a subclass of this subclass, the second layer further comprises a glidant. In a subclass of this subclass, the second layer further comprises a glidant, wherein the glidant is silicon dioxide.

In a class of this embodiment, the pharmaceutical composition comprises a second layer comprising: (i) about 5 to 15% by weight of atorvastatin; (ii) about 30 to 70% by weight of two diluents; (iii) about 0.1 to 15% of a binding agent; (iv) about 2 to 20% by weight of a disintegrant; (v) about 0.1 to 5% by weight of a surfactant, (vi) about 0.1 to 5% by weight of a lubricant; and (vi) about 2 to 50% by weight of an alkalizing agent.

In another class of this embodiment, the pharmaceutical composition comprises a second layer comprising: (i) about 5 to 15% by weight of a atorvastatin; (ii) about 15 to 40% by weight of the first diluent; (iii) about 15 to 40% by weight of the second diluent; (iv) about 0.1 to 15% of a binding agent; (v) about 2 to 20% by weight of a disintegrant; (vi) about 0.1 to 5% by weight of a surfactant, (vii) about 0.1 to 5% by weight of a lubricant; and (vi) about 2 to 50% by weight of an alkalizing agent.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of: magnesium stearate, and sodium stearyl fitmarate, or a mixture thereof; and the alkalizing agent is calcium carbonate. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. In a subclass of this subclass, the second layer further comprises a glidant. In a subclass of this subclass, the second layer further comprises a glidant, wherein the glidant is silicon dioxide.

In another class of this embodiment, the pharmaceutical composition comprises a second layer comprising: (i) about 5 to 15% by weight of atorvastatin; (ii) about 30 to 70% by weight of two diluents; (iii) about 0.1 to 15% of a binding agent; (iv) about 2 to 20% by weight of a disintegrant; (v) about 0.1 to 5% by weight of a surfactant, (vi) about 0.1 to 5% by weight of a lubricant; (vi) about 2 to 50% by weight of an alkalizing agent, and (vii) about 0.1 to 5% by weight of a glidant.

In another class of this embodiment, the pharmaceutical composition comprises a second layer comprising: (i) about 5 to 15% by weight of a atorvastatin; (ii) about 15 to 40% by weight of the first diluent; (iii) about 15 to 40% by weight of the second diluent; (iv) about 0.1 to 15% of a binding agent; (v) about 2 to 20% by weight of a disintegrant; (vi) about 0.1 to 5% by weight of a surfactant, (vii) about 0.1 to 5% by weight of a lubricant; (vi) about 2 to 50% by weight of an alkalizing agent, and (vii) about 0.1 to 5% by weight of a glidant.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of: magnesium stearate, and sodium stearyl fumarate, or a mixture thereof; the alkalizing agent is calcium carbonate; and the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; the alkalizing agent is calcium carbonate; and the glidant is silicon dioxide.

In another class of this embodiment, the second layer of the pharmaceutical composition (the atorvastatin layer) contains about 2 to 50% of an alkalizing agent. In a subclass of this class, the composition contains about 22 to 40% of an alkalizing agent. In another subclass of this class, the composition contains about 29 to 33% of an alkalizing agent. In another subclass of this class, the composition contains about 31% of an alkalizing agent. In another subclass of this class, the alkalizing agent is selected from the group consisting of L-arginine, calcium carbonate and sodium bicarbonate. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another embodiment of the present invention, atorvastatin is atorvastatin free acid. In another embodiment of the present invention, atorvastatin is selected from the group consisting of: atorvastatin, or a pharmaceutically acceptable salt, hydrate and solvate thereof. In another embodiment of the present invention, atorvastatin is selected from the group consisting of: atorvastatin, or a pharmaceutically acceptable salt and hydrate thereof. In another embodiment of the present invention, atorvastatin is selected from the group consisting of: atorvastatin calcium, or a hydrate thereof. In another embodiment of the present invention, atorvastatin is selected from the group consisting of: atorvastatin calcium salt (2:1), or a hydrate thereof. In another embodiment of the present invention, atorvastatin is selected from the group consisting of: atorvastatin calcium, or a trihydrate thereof. In another embodiment of the present invention, atorvastatin is selected from the group consisting of: atorvastatin calcium salt (2:1), or a trihydrate thereof. In a class of the embodiment of the present invention, atorvastatin calcium is amorphous. In another class of the embodiment of the present invention, atorvastatin calcium is crystalline. In another class of the embodiment of the present invention, atorvastatin calcium salt (2:1) is amorphous. In another class of the embodiment of the present invention, atorvastatin calcium salt (2:1) is crystalline. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

In a seventh embodiment of the present invention, the pharmaceutical composition comprises:

(a) a first layer comprising:

(i) about 25 to 35% by weight of a dipeptidyl peptidase-4 inhibitor, or a pharmaceutically acceptable salt thereof;

(ii) about 50-70% by weight of a diluent;

(iii) about 1-4% by weight of a disintegrant; and

(iv) about 0.5-10% by weight of a lubricant; and

(b) a second layer comprising:

(i) about 9 to 12% by weight of atorvastatin;

(ii) about 40 to 60% by weight of a diluent;

(iii) about 0.1 to 10% of a binding agent;

(iv) about 2 to 10% by weight of a disintegrant;

(v) about 0.1 to 3% by weight of a surfactant, and

(vi) about 0.1 to 1% by weight of a lubricant.

In a class of this embodiment, the pharmaceutical composition comprises a first layer comprising: (i) about 25 to 35% by weight of a dipeptidyl peptidase-4 inhibitor, or a pharmaceutically acceptable salt thereof; (ii) about 50-70% by weight of two diluents; (iii) about 1-4% by weight of a disintegrant; and (iv) about 1.5-10% by weight of two lubricants.

In another class of this embodiment, the pharmaceutical composition comprises a first layer comprising: (i) about 25 to 35% by weight of a dipeptidyl peptidase-4 inhibitor, or a pharmaceutically acceptable salt thereof; (ii) about 50-70% by weight of two diluents; (iii) about 1-4% by weight of a disintegrant; and (iv) about 1.5-10% by weight of two lubricants.

In another class of this embodiment, the first layer additionally comprises one or more excipients selected from the group consisting of: (i) about 20-40% by weight of a first diluent; (ii) about 20-40% of a second diluent; (iii) about 1-4% by weight of a disintegrant; (iv) about 0.25-5% by weight of a first lubricant and (v) about 0.25-5% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the pharmaceutical composition comprises a first layer wherein the dipeptidyl peptidase-4 inhibitor is sitagliptin, or a pharmaceutically acceptable salt thereof; the diluent is a mixture of microcrystalline cellulose and anhydrous dibasic calcium phosphate; the disintegrant is croscarmellose sodium; and the lubricant is a mixture of magnesium stearate and sodium stearyl fumarate.

In another class of this embodiment, the pharmaceutical composition comprises a second layer comprising: (i) about 9 to 12% by weight of atorvastatin; (ii) about 40 to 60% by weight of two diluents; (iii) about 0.1 to 10% of a binding agent; (iv) about 2 to 10% by weight of a disintegrant; (v) about 0.1 to 3% by weight of a surfactant, and (vi) about 0.1 to 1% by weight of a lubricant.

In another class of this embodiment, the pharmaceutical composition comprises a second layer comprising: (i) about 9 to 12% by weight of a atorvastatin; (ii) about 20 to 30% by weight of the first diluent; (iii) about 20 to 30% by weight of the second diluent; (iv) about 0.1 to 10% of a binding agent; (v) about 2 to 10% by weight of a disintegrant; (vi) about 0.1 to 3% by weight of a surfactant, and (vii) about 0.1 to 1% by weight of a lubricant.

In another subclass of this class the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; and the lubricant is selected from the group consisting of: magnesium stearate, and sodium stearyl fumarate, or a mixture thereof. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; and the lubricant is magnesium stearate. In a subclass of this subclass, the second layer further comprises a glidant. In a subclass of this subclass, the second layer further comprises a glidant, wherein the glidant is silicon dioxide.

In a class of this embodiment, the pharmaceutical composition comprises a second layer comprising: (i) about 9 to 12% by weight of atorvastatin; (ii) about 40 to 60% by weight of two diluents; (iii) about 0.1 to 10% of a binding agent; (iv) about 2 to 10% by weight of a disintegrant; (v) about 0.1 to 3% by weight of a surfactant, (vi) about 0.1 to 1% by weight of a lubricant; and (vi) about 22 to 40% by weight of an alkalizing agent.

In another class of this embodiment, the pharmaceutical composition comprises a second layer comprising: (i) about 9 to 12% by weight of a atorvastatin; (ii) about 20-30% by weight of the first diluent; (iii) about 20-30% by weight of the second diluent; (iv) about 0.1 to 10% of a binding agent; (v) about 2 to 10% by weight of a disintegrant; (vi) about 0.1 to 3% by weight of a surfactant, (vii) about 0.1 to 1% by weight of a lubricant; and (vi) about 22 to 40% by weight of an alkalizing agent.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of: magnesium stearate, and sodium stearyl fumarate, or a mixture thereof; and the alkalizing agent is calcium carbonate. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. In a subclass of this subclass, the second layer further comprises a glidant. In a subclass of this subclass, the second layer further comprises a glidant, wherein the glidant is silicon dioxide.

In a class of this embodiment, the pharmaceutical composition comprises a second layer comprising: (i) about 9 to 12% by weight of atorvastatin; (ii) about 40 to 60% by weight of two diluents; (iii) about 0.1 to 10% of a binding agent; (iv) about 2 to 10% by weight of a disintegrant; (v) about 0.1 to 3% by weight of a surfactant, (vi) about 0.1 to 1% by weight of a lubricant; (vi) about 22 to 40% by weight of an alkalizing agent, and (vii) about 0.1 to 2% by weight of a glidant.

In another class of this embodiment, the pharmaceutical composition comprises a second layer comprising: (i) about 9 to 12% by weight of a atorvastatin; (ii) about 20 to 30% by weight of the first diluent; (iii) about 20 to 30% by weight of the second diluent; (iv) about 0.1 to 10% of a binding agent; (v) about 2 to 10% by weight of a disintegrant; (vi) about 0.1 to 3% by weight of a surfactant, (vii) about 0.1 to 1% by weight of a lubricant (vi) about 22 to 40% by weight of an alkalizing agent, and (vii) about 0.1 to 2% by weight of a glidant.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of: magnesium stearate, and sodium stearyl fumarate, or a mixture thereof; the alkalizing agent is calcium carbonate; and the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; the alkalizing agent is calcium carbonate; and the glidant is silicon dioxide.

In another class of this embodiment, the second layer of the pharmaceutical composition (the atorvastatin layer) contains about 2 to 50% of an alkalizing agent. In a subclass of this class, the composition contains about 22 to 40% of an alkalizing agent. In another subclass of this class, the composition contains about 29 to 33% of an alkalizing agent. In another subclass of this class, the composition contains about 31% of an alkalizing agent. In another subclass of this class, the alkalizing agent is selected from the group consisting of L-arginine, calcium carbonate and sodium bicarbonate. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another embodiment of the present invention, atorvastatin is atorvastatin free acid. In another embodiment of the present invention, atorvastatin is selected from the group consisting of: atorvastatin, or a pharmaceutically acceptable salt, hydrate and solvate thereof. In another embodiment of the present invention, atorvastatin is selected from the group consisting of: atorvastatin, or a pharmaceutically acceptable salt and hydrate thereof. In another embodiment of the present invention, atorvastatin is selected from the group consisting of: atorvastatin calcium, or a hydrate thereof. In another embodiment of the present invention, atorvastatin is selected from the group consisting of: atorvastatin calcium salt (2:1), or a hydrate thereof. In another embodiment of the present invention, atorvastatin is selected from the group consisting of: atorvastatin calcium, or a trihydrate thereof. In another embodiment of the present invention, atorvastatin is selected from the group consisting of atorvastatin calcium salt (2:1), or a trihydrate thereof. In a class of the embodiment of the present invention, atorvastatin calcium is amorphous. In another class of the embodiment of the present invention, atorvastatin calcium is crystalline. In another class of the embodiment of the present invention, atorvastatin calcium salt (2:1) is amorphous. In another class of the embodiment of the present invention, atorvastatin calcium salt (2:1) is crystalline. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

In an eighth embodiment of the present invention, the pharmaceutical composition comprises:

(a) a first layer comprising:

(i) about 30 to 35% by weight of a dipeptidyl peptidase-4 inhibitor, or a pharmaceutically acceptable salt thereof;

(ii) about 50-70% by weight of a diluent;

(iii) about 1-4% by weight of a disintegrant; and

(iv) about 0.5-7% by weight of a lubricant; and

(b) a second layer comprising:

(i) about 10 to 11% by weight of atorvastatin;

(ii) about 48 to 50% by weight of a diluent;

(iii) about 1 to 3% of a binding agent;

(iv) about 5 to 7% by weight of a disintegrant;

(v) about 0.1 to 1% by weight of a surfactant, and

(vi) about 0.3 to 0.7% by weight of a lubricant.

In a class of this embodiment, the pharmaceutical composition comprises a first layer comprising: (i) about 30 to 35% by weight of a dipeptidyl peptidase-4 inhibitor, or a pharmaceutically acceptable salt thereof; (ii) about 50-70% by weight of two diluents; (iii) about 1-4% by weight of a disintegrant; and (iv) about 0.5-7% by weight of two lubricants.

In another class of this embodiment, the first layer additionally comprises one or more excipients selected from the group consisting of (i) about 20-40% by weight of a first diluent; (ii) about 20-40% of a second diluent; (iii) about 1-4% by weight of a disintegrant; (iv) about 0.25-5% by weight of a first lubricant and (v) about 0.25-5% by weight of a second lubricant. In a subclass of this class, the first diluent is anhydrous dibasic calcium phosphate; the second diluent is microcrystalline cellulose; the disintegrant is croscarmellose sodium; the first lubricant is sodium stearyl fumarate; and the second lubricant is magnesium stearate.

In another class of this embodiment, the pharmaceutical composition comprises a first layer wherein the dipeptidyl peptidase-4 inhibitor is sitagliptin, or a pharmaceutically acceptable salt thereof; the diluent is a mixture of microcrystalline cellulose and anhydrous dibasic calcium phosphate; the disintegrant is croscarmellose sodium; and the lubricant is a mixture of magnesium stearate and sodium stearyl fumarate.

In a class of this embodiment, the pharmaceutical composition comprises a second layer comprising: (i) about 10 to 11% by weight of atorvastatin; (ii) about 48 to 50% by weight of two diluents; (iii) about 1 to 3% of a binding agent; (iv) about 5 to 7% by weight of a disintegrant; (v) about 0.1 to 1% by weight of a surfactant, and (vi) about 0.3 to 0.7% by weight of a lubricant.

In another class of this embodiment, the pharmaceutical composition comprises a second layer comprising: (1) about 10 to 11% by weight of a atorvastatin; (ii) about 20 to 30% by weight of the first diluent; (iii) about 20 to 30% by weight of the second diluent; (iv) about 1 to 3% of a binding agent; (v) about 5 to 7% by weight of a disintegrant; (vi) about 0.1 to 1% by weight of a surfactant, and (vii) about 0.3 to 0.7% by weight of a lubricant.

In a subclass of this class the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; and the lubricant is selected from the group consisting of magnesium stearate, and sodium stearyl fumarate, or a mixture thereof. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; and the lubricant is magnesium stearate. In a subclass of this subclass, the second layer further comprises a glidant. In a subclass of this subclass, the second layer further comprises a glidant, wherein the glidant is silicon dioxide.

In a class of this embodiment, the pharmaceutical composition comprises a second layer comprising: (i) about 10 to 11% by weight of atorvastatin; (ii) about 48 to 50% by weight of two diluents; (iii) about 1 to 3% of a binding agent; (iv) about 5 to 7% by weight of a disintegrant; (v) about 0.1 to 1% by weight of a surfactant, (vi) about 0.3 to 0.7% by weight of a lubricant; and (vi) about 29 to 33% by weight of an alkalizing agent.

In another class of this embodiment, the pharmaceutical composition comprises a second layer comprising: (i) about 10 to 11% by weight of a atorvastatin; (ii) about 20 to 30% by weight of the first diluent; (iii) about 20 to 30% by weight of the second diluent; (iv) about 1 to 3% of a binding agent; (v) about 5 to 7% by weight of a disintegrant; (vi) about 0.1 to 1% by weight of a surfactant, (vii) about 0.3 to 0.7% by weight of a lubricant; and (vi) about 29 to 33% by weight of an alkalizing agent.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of: magnesium stearate, and sodium stearyl fumarate, or a mixture thereof; and the alkalizing agent is calcium carbonate. In a subclass of this subclass, the second layer further comprises a glidant. In a subclass of this subclass, the second layer further comprises a glidant, wherein the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. In a subclass of this subclass, the second layer further comprises a glidant. In a subclass of this subclass, the second layer further comprises a glidant, wherein the glidant is silicon dioxide.

In a class of this embodiment, the pharmaceutical composition comprises a second layer comprising: (i) about 10 to 11% by weight of atorvastatin; (ii) about 48 to 50% by weight of two diluents; (iii) about 1 to 3% of a binding agent; (iv) about 5 to 7% by weight of a disintegrant; (v) about 0.1 to 1% by weight of a surfactant, (vi) about 0.3 to 0.7% by weight of a lubricant; (vi) about 29 to 33% by weight of an alkalizing agent, and (vii) about 0.1 to 1% by weight of a glidant.

In another class of this embodiment, the pharmaceutical composition comprises a second layer comprising: (i) about 10 to 11% by weight of a atorvastatin; (ii) about 20 to 30% by weight of the first diluent; (iii) about 20 to 30% by weight of the second diluent; (iv) about 1 to 3% of a binding agent; (v) about 5 to 7% by weight of a disintegrant; (vi) about 0.1 to 1% by weight of a surfactant, (vii) about 0.3 to 0.7% by weight of a lubricant; (vi) about 29 to 33% by weight of an alkalizing agent, and (vii) about 0.1 to 1% by weight of a glidant.

In a subclass of this class, the diluent in the second layer is selected from the group consisting of: microcrystalline cellulose, lactose, lactose monohydrate and mannitol, or a mixture thereof; the binding agent is hydroxypropyl cellulose; the disintegrant is croscarmellose sodium; the surfactant is polysorbate or polysorbate 80; the lubricant is selected from the group consisting of: magnesium stearate, and sodium stearyl fumarate, or a mixture thereof; the alkalizing agent is calcium carbonate; and the glidant is silicon dioxide.

In another subclass of this class, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; the alkalizing agent is calcium carbonate; and the glidant is silicon dioxide.

In another class of this embodiment, the second layer of the pharmaceutical composition (the atorvastatin layer) contains about 2 to 50% of an alkalizing agent. In a subclass of this class, the composition contains about 22 to 40% of an alkalizing agent. In another subclass of this class, the composition contains about 29 to 33% of an alkalizing agent. In another subclass of this class, the composition contains about 31% of an alkalizing agent. In another subclass of this class, the alkalizing agent is selected from the group consisting of L-arginine, calcium carbonate and sodium bicarbonate. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of this embodiment, the dipeptidyl peptidase-4 inhibitor is selected from the group consisting of: alogliptin, carmegiptin, denagliptin, dutogliptin, linagliptin, melogliptin, saxagliptin, sitagliptin, and vildagliptin, or a pharmaceutically acceptable salt of each thereof. In another class of this embodiment, the dipeptidyl peptidase-4 inhibitor is selected from the group consisting of sitagliptin, vildagliptin, and saxagliptin, or a pharmaceutically acceptable salt of each thereof. In a subclass of this class, the dipeptidyl peptidase-4 inhibitor is sitagliptin, or the dihydrogen phosphate salt thereof.

In another embodiment of the present invention, atorvastatin is atorvastatin free acid. In another embodiment of the present invention, atorvastatin is selected from the group consisting of: atorvastatin, or a pharmaceutically acceptable salt, hydrate and solvate thereof. In another embodiment of the present invention, atorvastatin is selected from the group consisting of: atorvastatin, or a pharmaceutically acceptable salt and hydrate thereof. In another embodiment of the present invention, atorvastatin is selected from the group consisting of: atorvastatin calcium, or a hydrate thereof. In another embodiment of the present invention, atorvastatin is selected from the group consisting of: atorvastatin calcium salt (2:1), or a hydrate thereof. In another embodiment of the present invention, atorvastatin is selected from the group consisting of: atorvastatin calcium, or a trihydrate thereof. In another embodiment of the present invention, atorvastatin is selected from the group consisting of atorvastatin calcium salt (2:1), or a trihydrate thereof. In a class of the embodiment of the present invention, atorvastatin calcium is amorphous. In another class of the embodiment of the present invention, atorvastatin calcium is crystalline. In another class of the embodiment of the present invention, atorvastatin calcium salt (2:1) is amorphous. In another class of the embodiment of the present invention, atorvastatin calcium salt (2:1) is crystalline. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

In another class of the embodiments of the present invention, the pharmaceutical composition contains about 20 to 45% by weight of sitagliptin dihydrogen phosphate. In a subclass of this class, the composition contains about 25 to 35% of sitagliptin dihydrogen phosphate. In another subclass of this class, the composition contains about 32 to 33% of sitagliptin dihydrogen phosphate. In another subclass of this class, the composition contains about 32.13% of sitagliptin dihydrogen phosphate.

In another class of the embodiments of the present invention, the pharmaceutical composition contains about 25 to 45% by weight of sitagliptin, or a pharmaceutically acceptable salt thereof. In a subclass of this class, the composition contains about 25 to 35% of sitagliptin, or a pharmaceutically acceptable salt thereof. In another subclass of this class, the composition contains about 32 to 33% of sitagliptin, or a pharmaceutically acceptable salt thereof. In another subclass of this class, the composition contains about 32.13% of sitagliptin, or a pharmaceutically acceptable salt thereof.

In another class of the embodiments of the present invention, the pharmaceutical composition contains about 5 to 15% by weight of atorvastatin. In a subclass of this class, the composition contains about 8 to 12% of atorvastatin. In another subclass of this class, the composition contains about 9 to 11% of atorvastatin. In another subclass of this class, the composition contains about 9 to 12% of atorvastatin. In another subclass of this class, the composition contains about 10 to 11% of atorvastatin. In another subclass of this class, the composition contains about 10.34% of atorvastatin. In another subclass of this class, the composition contains about 10.50% of atorvastatin. In another subclass of this class, the composition contains about 10.55% of atorvastatin.

In another class of the embodiments of the present invention, the first layer of the pharmaceutical composition (the sitagliptin layer) contains about 40 to 80% by weight of a diluent. In a subclass of this class, the composition contains about 50 to 70% of a diluent. In another subclass of this class, the composition contains about 60-63% of a diluent. In another subclass of this class, the composition contains about 61.88% of a diluent. In another subclass of this class, the composition contains about 10 to 60% of a first diluent; and contains about 10 to 60% of a second diluent. In another subclass of this class, the composition contains about 20 to 40% of a first diluent; and contains about 20 to 40% of a second diluent. In another subclass of this class, the composition contains about 30 to 31% of a first diluent; and contains about 30 to 31% of a second diluent. In another subclass of this class, the composition contains about 30.94% of a first diluent. In another subclass of this class, the composition contains about 30.94% of a second diluent. In another subclass of this class, the diluent is microcrystalline cellulose or anhydrous dibasic calcium phosphate. In another subclass of this class, the diluent is microcrystalline cellulose and anhydrous dibasic calcium phosphate. In another subclass of this class, first diluent is dibasic calcium phosphate and the second diluent is microcrystalline cellulose.

In another class of the embodiments of the present invention, the second layer of the pharmaceutical composition (the atorvastatin layer) contains about 2 to 90% by weight of a diluent. In a subclass of this class, the composition contains about 10 to 90% by weight of a diluent. In another subclass of this class, the composition contains about 50 to 90% by weight of a diluent. In another subclass of this class, the composition contains about 60 to 80% by weight of a diluent. In another subclass of this class, the composition contains about 72 to 76% by weight of a diluent. In another subclass of this class, the composition contains about 74.16% by weight of a diluent. In another subclass of this class, the composition contains about 30 to 70% by weight of a diluent. In another subclass of this class, the composition contains about 40 to 60% by weight of a diluent. In another subclass of this class, the composition contains about 48 to 50% by weight of a diluent. In another subclass of this class, the composition contains about 49.1% by weight of a diluent. In another subclass of this class, the diluent is microcrystalline cellulose, mannitol, lactose or anhydrous lactose, or a mixture thereof. In another subclass of this class, the diluent is a mixture of microcrystalline cellulose and anhydrous lactose, or a mixture of microcrystalline cellulose and mannitol. In another subclass of this class, the diluent is a 1:1 mixture of microcrystalline cellulose and anhydrous lactose, or a 1:1 mixture of microcrystalline cellulose and mannitol. In another subclass of this class, the diluent is a mixture of microcrystalline cellulose and lactose monohydrate.

In another class of the embodiments of the present invention, the first layer of the pharmaceutical composition (the sitagliptin layer) contains about 0.1-10% by weight of a disintegrant. In a subclass of this class, the composition contains about 0.5 to 10% of a disintegrant. In a subclass of this class, the composition contains about 2 to 10% of a disintegrant. In another subclass of this class, the composition contains about 1 to 10% of a disintegrant. In another subclass of this class, the composition contains about 1 to 4% of a disintegrant. In another subclass of this class, the composition contains about 1 to 3% of a disintegrant. In another subclass of this class, the composition contains about 2% of a disintegrant. In another subclass of this class, the disintegrant is croscarmellose sodium.

In another class of the embodiments of the present invention, the second layer of the pharmaceutical composition (the atorvastatin layer) contains about 0.1 to 20% by weight of a disintegrant. In a subclass of this class, the composition contains about 2 to 20% of a disintegrant. In a subclass of this class, the composition contains about 2 to 10% of a disintegrant. In another subclass of this class, the composition contains about 5 to 7% of a disintegrant. In another subclass of this class, the composition contains about 6% of a disintegrant. In another subclass of this class, the disintegrant is croscarmellose sodium.

In another class of the embodiments of the present invention, the second layer of the pharmaceutical composition (the atorvastatin layer) contains about 0 to 5% by weight of a surfactant. In a subclass of this class, the composition contains about 0 to 3% of a surfactant. In another subclass of this class, the composition contains about 0 to 2% of a surfactant. In another subclass of this class, the composition contains about 1% of a surfactant. In another subclass of this class, the composition contains about 0.1 to 5% by weight of a surfactant. In another subclass of this class, the composition contains about 0.1 to 3% by weight of a surfactant. In another subclass of this class, the composition contains about 0.1 to 1% by weight of a surfactant. In another subclass of this class, the composition contains about 0.4% of a surfactant. In another subclass of this class, the surfactant is sodium lauryl sulfate. In another subclass of this class, the surfactant is polysorbate. In another subclass of this class, the surfactant is polysorbate 80.

In another class of the embodiments of the present invention, the second layer of the pharmaceutical composition (the atorvastatin layer) contains about 0.1 to 4,% by weight of an alkalizing agent. In a subclass of this class, the composition contains less than 5% of an alkalizing agent. In a subclass of this class, the composition contains less than 5% of an alkalizing agent selected from the group consisting of L-arginine and sodium bicarbonate. In another subclass of this class, the composition contains less than 5% of an alkalizing agent selected from the group consisting of L-arginine, calcium carbonate and sodium bicarbonate. In a subclass of this class, the composition contains less than 5% L-arginine. In another subclass of this class, the composition contains less than 5% sodium bicarbonate. In another subclass of this class, the composition contains about 0 to 4% of an alkalizing agent. In another subclass of this class, the composition contains about 0.2 to 4% of an alkalizing agent. In another subclass of this class, the composition contains about 1 to 4% of an alkalizing agent. In a subclass of this class, the composition contains about 2 to 4% of an alkalizing agent. In a subclass of this class, the composition contains about 3 to 4% of an alkalizing agent. In another subclass of this class, the composition contains about 4% of an alkalizing agent. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of the embodiments of the present invention, the composition contains 2 to 50% of an alkalizing agent. In a subclass of this class, the composition contains about 22 to 40% of an alkalizing agent. In another subclass of this class, the composition contains about 29 to 33% of an alkalizing agent. In another subclass of this class, the composition contains about 31% of an alkalizing agent. In another subclass of this class, the alkalizing agent is selected from the group consisting of L-arginine and sodium bicarbonate. In another subclass of this class, the alkalizing agent is L-arginine. In another subclass of this class, the alkalizing agent is sodium bicarbonate. In another subclass of this class, the alkalizing agent is calcium carbonate.

In another class of the embodiments of the present invention, the first layer of the pharmaceutical composition (the sitagliptin layer) contains about 0.5 to 10% by weight of a lubricant. In a subclass of this class, the composition contains about 1.5 to 7% of a lubricant. In another subclass of this class, the composition contains about 0.5 to 5% of a lubricant. In another subclass of this class, the composition contains about 0.25 to 5% of a first lubricant; and contains about 0.25 to 5% of a second lubricant. In another subclass of this class, the composition contains about 0.5 to 5% of a first lubricant; and contains about 0.5 to 5% of a second lubricant. In another subclass of this class, the composition contains about 2 to 4% of a first lubricant; and contains about 0.5 to 1.5% of a second lubricant. In another subclass of this class, the composition contains about 3% of a first lubricant; and contains about 1% of a second lubricant. In another subclass of this class, the lubricant is a mixture of sodium stearyl fumarate or magnesium stearate. In another subclass of this class, the lubricant is sodium stearyl fumarate and magnesium stearate. In another subclass of this class, the first lubricant is sodium stearyl fumarate. In another subclass of this class, the second lubricant is magnesium stearate.

In another class of the embodiments of the present invention, the second layer of the pharmaceutical composition (the atorvastatin layer) contains about 0.25 to 10% by weight of a lubricant. In a subclass of this class, the composition contains about 0.25 to 5% by weight of a lubricant. In a subclass of this class, the composition contains about 0.5 to 3% by weight of a lubricant. In a subclass of this class, the composition contains about 1 to 3% by weight of a lubricant. In another subclass of this class, the composition contains about 2.5% by weight of a lubricant. In another subclass of this class, the composition contains about 0.1 to 5% by weight of a lubricant. In another subclass of this class, the composition contains about 0.1 to 1% by weight of a lubricant. In another subclass of this class, the composition contains about 0.3 to 0.7% by weight of a lubricant. In another subclass of this class, the composition contains about 0.5% by weight of a lubricant. In another subclass of this class, the lubricant is sodium stearyl fumarate or magnesium stearate. In another subclass of this class, the lubricant is a mixture of sodium stearyl fumarate and magnesium stearate. In another subclass of this class, the lubricant is sodium stearyl fumarate. In another subclass of this class, the lubricant is magnesium stearate.

In another class of the embodiments of the present invention, the second layer of the pharmaceutical composition (the atorvastatin layer) contains about 0 to 15% by weight of a binding agent. In a subclass of this class, the composition contains about 0 to 10% by weight of a binding agent. In a subclass of this class, the composition contains about 1 to 5% by weight of a binding agent. In another subclass of this class, the composition contains about 2 to 4% by weight of a binding agent. In another subclass of this class, the composition contains about 3% by weight of a binding agent. In a subclass of this class, the composition contains about 0.1 to 10% by weight of a binding agent. In a subclass of this class, the composition contains about 1 to 3% by weight of a binding agent. In another subclass of this class, the composition contains about 2% by weight of a binding agent. In another subclass of this class, the binding agent is hydroxypropylcellulose, polyvinylpyrrolidone, pregelatinized starch or pregelatinized corn starch, or a mixture thereof. In another subclass of this class, the binding agent is hydroxypropylcellulose.

In another embodiment of the present invention, the first layer of the pharmaceutical composition (the DPP-4/sitagliptin layer) contains about 0 to 5% of a glidant. In another embodiment of the present invention, the first layer of the pharmaceutical composition (the DPP-4/sitagliptin layer) contains about 0.1 to 5% of a glidant. In another embodiment of the present invention, the first layer of the pharmaceutical composition (the DPP-4/sitagliptin layer) contains about 0 to 2% of a glidant. In another embodiment of the present invention, the first layer of the pharmaceutical composition (the DPP-4/sitagliptin layer) contains about 0.1 to 2% of a glidant. In another embodiment of the present invention, the first layer of the pharmaceutical composition (the DPP-4/sitagliptin layer) contains about 0 to 1% of a glidant. In another embodiment of the present invention, the first layer of the pharmaceutical composition (the DPP-4/sitagliptin layer) contains about 0.1 to 1% of a glidant. In another embodiment of the present invention, the first layer of the pharmaceutical composition (the DPP-4/sitagliptin layer) contains about 0.25 to 1% of a glidant. In another embodiment of the present invention, the first layer of the pharmaceutical composition (the DPP-4/sitagliptin layer) contains about 0.25%, 0.5%, 1% or 2% of a glidant. In class of these embodiments, the glidant is silicon dioxide.

In another embodiment of the present invention, the second layer of the pharmaceutical composition (the atorvastatin layer) contains about 0 to 5% of a glidant. In another embodiment of the present invention, the second layer of the pharmaceutical composition (the atorvastatin layer) contains about 0.1 to 5% of a glidant. In another embodiment of the present invention, the second layer of the pharmaceutical composition (the atorvastatin layer) contains about 0 to 2% of a glidant. In another embodiment of the present invention, the second layer of the pharmaceutical composition (the atorvastatin layer) contains about 0.1 to 2% of a glidant. In another embodiment of the present invention, the second layer of the pharmaceutical composition (the atorvastatin layer) contains about 0 to 1% of a glidant. In another embodiment of the present invention, the second layer of the pharmaceutical composition (the atorvastatin layer) contains about 0.1 to 1% of a glidant. In another embodiment of the present invention, the second layer of the pharmaceutical composition (the atorvastatin layer) contains about 0.25 to 1 of a glidant. In another embodiment of the present invention, the second layer of the pharmaceutical composition (the atorvastatin layer) contains about 0.5% of a glidant. In another embodiment of the present invention, the second layer of the pharmaceutical composition (the atorvastatin layer) contains about 0.25%, 0.5%, 1%, 2% or 4% of a glidant. In another embodiment of the present invention, the second layer of the pharmaceutical composition (the atorvastatin layer) contains about 0.26%, 0.52%, 1.04%, 2.08 or 4.16% of a glidant. In class of these embodiments, the glidant is silicon dioxide.

In further embodiments of the present invention, the pharmaceutical compositions are envisioned for commercial development:

### Tablets of 50 mg Dipeptidyl Peptidase-4 Inhibitor/5 mg Atorvastatin Potency

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight of the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second layer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydtoxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

### Tablets of 50 Mg Dipeptidyl Peptidase-4 Inhibitor/10 Mg Atorvastatin Potency

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight of the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second layer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

### Tablets of 50 Mg Dipeptidyl Peptidase-4 Inhibitor/20 Mg Atorvastatin Potency

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight of the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second layer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

### Tablets of 50 Mg Dipeptidyl Peptidase-4 Inhibitor/40 Mg Atorvastatin Potency

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight of the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second layer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of: atorvastatin calcium salt (2:1), or a trihydrate thereof. In another class of this embodiment, atorvastatin is selected from the group consisting of amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

### Tablets of 50 Mg Dipeptidyl Peptidase-4 Inhibitor/80 Mg Atorvastatin Potency:

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight of the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second layer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of: atorvastatin calcium salt (2:1), or a trihydrate thereof. In another class of this embodiment, atorvastatin is selected from the group consisting of amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

### Tablets of 100 mg Dipeptidyl Peptidase-4 Inhibitor/5 mg Atorvastatin Potency:

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight of the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second layer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

### Tablets of 100 Mg Dipeptidyl Peptidase-4 Inhibitor/10 Mg Atorvastatin Potency:

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight or the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second layer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

### Tablets of 100 mg Dipeptidyl Peptidase-4 Inhibitor/20 mg Atorvastatin Potency:

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight of the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second layer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of: atorvastatin calcium salt (2:1), or a trihydrate thereof. In another class of this embodiment, atorvastatin is selected from the group consisting of amorphous atorvastatin calcium, or a hydrate or solvate thereof. In a subclass of this class, atorvastatin is amorphous atorvastatin calcium salt (2:1). In another subclass of this class, atorvastatin is selected from the group consisting of; amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

### Tablets of 100 Mg Dipeptidyl Peptidase-4 Inhibitor/40 Mg Atorvastatin Potency:

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight of the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second Myer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form 1.

### Tablets of 100 Mg Dipeptidyl Peptidase-4 Inhibitor/80 Mg Atorvastatin Potency:

For the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.34% by weight of the second layer of atorvastatin, about 74 to 75% by weight of the second layer of a diluent, about 3% by weight of the second layer of a binding agent; about 6% by weight of the second layer of disintegrant; about 0-4% by weight of the second layer of an alkalizing agent; about 1% by weight of the second layer of surfactant; and about 1.5% by weight of the second layer of a lubricant. The first layer may also contain silicon dioxide. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and mannitol, or a mixture of microcrystalline cellulose and anhydrous lactose; the binding agent is hydroxypropyl cellulose; the disintegrant is croscarmellose sodium; the surfactant is sodium lauryl sulfate; and the lubricant is magnesium stearate. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of: amorphous atorvastatin calcium salt (2:1), or a hydrate thereof.

Alternatively, for the first layer: about 32.13% by weight of the first layer of the dipeptidyl peptidase-4 inhibitor or a pharmaceutically acceptable salt thereof; about 61-62% by weight of the first layer of a diluent; about 2% by weight of the first layer of a disintegrant; and about 4% by weight of the first layer of a lubricant. For the second layer: about 10.50 to 10.55% by weight of the second layer of atorvastatin, about 49.10 to 49.33% by weight of the second layer of a diluent, about 2.00 to 2.01% by weight of the second layer of a binding agent; about 6.00 to 6.03% by weight of the second layer of disintegrant; about 31.00 to 31.16% by weight of the second layer of an alkalizing agent; about 0.40% by weight of the second layer of surfactant; and about 0.5% by weight of the second layer of a lubricant. The second layer may also contain 0 to 5% of a glidant. In a class of this embodiment, the dipeptidyl peptidase-4 inhibitor is sitagliptin, or a pharmaceutically acceptable salt thereof. In another class of this embodiment, the dipeptidyl peptidase-4 inhibitor is selected from the group consisting of: alogliptin, carmegliptin, melogliptin, dutogliptin, denagliptin, linagliptin, saxagliptin and vildagliptin, or a pharmaceutically acceptable salt thereof. In another class of this embodiment, the diluent in the first layer is a mixture of dibasic anhydrous calcium phosphate and microcrystalline cellulose; the disintegrant in the first layer is sodium croscarmellose; the lubricant in the first layer is a mixture of sodium stearyl fumarate and magnesium stearate. In another class of this embodiment, the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate; and the alkalizing agent is calcium carbonate. The second layer may also contain silicon dioxide as a glidant. In another class of this embodiment, atorvastatin is selected from the group consisting of: atorvastatin calcium salt (2:1), or a trihydrate thereof. In another class of this embodiment, atorvastatin is selected from the group consisting of: amorphous atorvastatin calcium, or a hydrate or solvate thereof. In a subclass of this class, atorvastatin is amorphous atorvastatin calcium salt (2:1). In another subclass of this class, atorvastatin is selected from the group consisting of amorphous atorvastatin calcium salt (2:1), or a hydrate thereof. In another class of this embodiment, atorvastatin is atorvastatin calcium salt (2:1) trihydrate. In another class of this embodiment, atorvastatin is crystalline atorvastatin calcium salt (2:1) trihydrate. In another class of this embodiment, atorvastatin is crystalline atorvastatin calcium salt (2:1) trihydrate crystal form I.

The pharmaceutical tablet compositions of the present invention may also contain one or more additional formulation ingredients selected from a wide variety of excipients known in the pharmaceutical formulation art. According to the desired properties of the pharmaceutical composition, any number of ingredients may be selected, alone or in combination, based upon their known uses in preparing tablet compositions. Such ingredients include, but are not limited to, diluents, compression aids, glidants, disintegrants, lubricants, flavors, flavor enhancers, sweeteners, and preservatives.

The term “tablet” as used herein is intended to encompass compressed pharmaceutical dosage formulations of all shapes and sizes, whether coated or uncoated. Substances which may be used for coating include hydroxypropylcellulose, hydroxypropylmethylcellulose, titanium dioxide, talc, sweeteners, colorants, and flavoring agents.

The term and symbol “% by weight” and “%” as used herein refers to the percentage by weight of the excipient and active ingredient (DPP-4 inhibitor or atorvastatin) in each individual layer in the bilayer tablet, wherein the “individual layer” means the first layer or the second layer of the bilayer tablet.

Also provided is a method for treating hypercholesterolemia (heterozygous familial and nonfamilial), mixed dyslipidemia (Fredrickson Types IIa and IIb), homozygous familial hypercholesterolemia or a combination thereof as well as treating heterozygous familial hypercholesterolemia in pediatric patients 10-17 years of age, in a patient by administering to the patient a therapeutically effective amount of a composition of the present invention. When an additional active agent is present in the composition, there is also provided a method of treatment for disorders in addition to those noted above, such as diabetes, obesity, etc., which will depend on the selection of the active agent for co-administration.

The first layer (the DPP-4 inhibitor or sitagliptin layer) can be the layer at the bottom of the bilayer tablet or at the top of the bilayer tablet (filled into the die either first or second). The second layer (the atorvastatin layer) can be the layer at the bottom of the bilayer tablet or at the top of the bilayer tablet (filled into the die either first or second).

The present invention provides a fixed dose combination of a dipeptidyl peptidase-4 (DPP-4) inhibitor, including but not limited to sitagliptin, or a pharmaceutically acceptable salt thereof, and atorvastatin, or a pharmaceutically acceptable salt thereof, in which both drugs are stable in a single tablet. More particularly, the present invention provides a fixed dose combination comprised of a layer of a dipeptidyl peptidase-4 (DPP-4) inhibitor, including but not limited to sitagliptin, or a pharmaceutically acceptable salt thereof, and a layer of atorvastatin in a single bilayer tablet.

The present invention also provides methods for treating Type 2 diabetes and hypercholesterolemia by orally administering to a host in need of such treatment a therapeutically effective amount of one of the fixed-dose combination pharmaceutical compositions of the present invention. In one embodiment the patient in need of such treatment is a human. In another embodiment the pharmaceutical composition is in the dosage form of a tablet. The pharmaceutical compositions comprising the fixed-dose combination may be administered once-daily (QD), twice-daily (BID), or thrice-daily (TID).

The following examples further describe and demonstrate embodiments within the scope of the present invention. The examples are given solely for the purpose of illustration and are not intended to be construed as limitations of the present invention as many variations thereof are possible without departing from the spirit and scope of the invention.

### Example 1

### Bilayer Tablet Formulation

### General Composition

| | | % of | |
|---|---|---|---|
| | Amount | each | |
| Component | (mg/tablet) | layer | range |
| Layer 1 | | | |
| Sitagliptin phosphate^{†} | 128.5 | 32.13 | 50 and 100 mg |
| Calcium Phosphate, dibasic | 123.75 | 30.94 | 10-60% (of layer 1) |
| Microcrystalline cellulose | 123.75 | 30.94 | 10-60% (of layer 1) |
| or | | | |
| Silicified Microcrystalline | | | |
| Cellulose | | | |
| Croscarmellose sodium | 8 | 2.00 | 0.1-10% (of layer 1) |
| Sodium stearyl fumarate | 12 | 3.00 | 0.25-5% (of layer 1) |
| Magnesium stearate | 4 | 1.00 | 0.25-5% (of layer 1) |
| optional silicon dioxide | 2.48 | 0.62 | microcrystalline |
| | | | 0-5% (of amount of |
| | | | cellulose in layer 1) |
| Total Layer 1 weight | 400 | 100 | |
| Layer 2 | | | |
| Atorvastatin calcium^{‡} | 82.72 | 10.34 | 5, 10, 20, 40, 80 mg |
| Microcrystalline cellulose | 296.64 | 37.08 | 2-60% (of layer 2) |
| Mannitol or lactose | 296.64 | 37.08 | 10-90% (of layer 2) |
| Hydroxypropyl cellulose | 24 | 3 | 0-15% (of layer 2) |
| Croscarmellose sodium | 48 | 6 | 2-20% (of layer 2) |
| Sodium bicarbonate | 32 | 4 | 0-4% (of layer 2) |
| Sodium lauryl sulfate | 8 | 1 | 0-5% (of layer 2) |
| Magnesium stearate | 12 | 1.5 | 0.1-5% (of layer 2) |
| Total Layer 2 weight | 800 | 100 | |
| Core Bilayer Tablet Weight | 1200 | | |
| (Layer 1 + Layer 2) | | | |
| Total Film Coating | 48 | 4 | 2-10% (of tablet |
| | | | weight) |

^{†}Salt factor: 1.285 (sitagliptin)
^{‡}Salt factor: 1.034 (atorvastatin)

### Example 2

### Bilayer Tablet Formulations Comprising a 100 mg Sitagliptin Phosphate Layer and an Atorvastatin Mannitol (A1) Layer

| | 100/10 | 100/20 | 100/40 | |
|---|---|---|---|---|
| | potency | potency | potency | 100/80 |
| | (mg/ | (mg/ | (mg/ | potency |
| Component | tablet) | tablet) | tablet) | (mg/tablet) |
| Layer 1 | | | | |
| Sitagliptin phosphate^{†} | 128.5 | 128.5 | 128.5 | 128.5 |
| Calcium Phosphate, dibasic | 123.75 | 123.75 | 123.75 | 123.75 |
| Microcrystalline cellulose or | 123.75 | 123.75 | 123.75 | 123.75 |
| silicified Microcrystalline | | | | |
| cellulose | | | | |
| Croscarmellose sodium | 8 | 8 | 8 | 8 |
| Sodium stearyl fumarate | 12 | 12 | 12 | 12 |
| Magnesium stearate | 4 | 4 | 4 | 4 |
| Total Layer 1 weight | 400 | 400 | 400 | 400 |
| Layer 2 | | | | |
| Atorvastatin Calcium^{‡} | 10.34 | 20.68 | 41.36 | 82.72 |
| Microcrystalline cellulose | 37.08 | 74.16 | 148.32 | 296.64 |
| Mannitol | 37.08 | 74.16 | 148.32 | 296.64 |
| Hydroxypropyl cellulose | 3 | 6 | 12 | 24 |
| Croscarmellose sodium | 6 | 12 | 24 | 48 |
| Sodium bicarbonate | 4 | 8 | 16 | 32 |
| Sodium lauryl sulfate | 1 | 2 | 4 | 8 |
| Magnesium stearate | 1.5 | 3 | 6 | 12 |
| Total Layer 2 weight | 100 | 200 | 400 | 800 |
| Core Bilayer Tablet Weight | 500 | 600 | 800 | 1200 |
| (Layer 1 + Layer 2) | | | | |
| Total Film Coating | 20 | 24 | 32 | 48 |

^{†}Salt factor: 1.285 (sitagliptin),
^{‡}Salt factor: 1.034 (atorvastatin)

### Example 3

### Bilayer Tablet Formulations Comprising a 50 mg Sitagliptin Phosphate Layer and an Atorvastatin Mannitol (A1) Layer

| | 50/10 | 50/20 | 50/40 |
|---|---|---|---|
| | potency | potency | potency |
| Component | (mg/tablet) | (mg/tablet) | (mg/tablet) |
| Layer 1 | | | |
| Sitagliptin phosphate^{†} | 64.25 | 64.25 | 64.25 |
| Calcium Phosphate, dibasic | 61.875 | 61.875 | 61.875 |
| Microcrystalline cellulose | 61.875 | 61.875 | 61.875 |
| or silicified | | | |
| Microcrystalline cellulose | | | |
| Croscarmellose sodium | 4 | 4 | 4 |
| Sodium stearyl fumarate | 6 | 6 | 6 |
| Magnesium stearate | 2 | 2 | 2 |
| Total Layer 1 weight | 200 | 200 | 200 |
| Layer 2 | | | |
| Atorvastatin Calcium^{‡} | 10.34 | 20.68 | 41.36 |
| Microcrystalline cellulose | 37.08 | 74.16 | 148.32 |
| Mannitol | 37.08 | 74.16 | 148.32 |
| Hydroxypropyl cellulose | 3 | 6 | 12 |
| Croscarmellose sodium | 6 | 12 | 24 |
| Sodium bicarbonate | 4 | 8 | 16 |
| Sodium lauryl sulfate | 1 | 2 | 4 |
| Magnesium stearate | 1.5 | 3 | 6 |
| Total Layer 2 weight | 100 | 200 | 400 |
| Core Bilayer Tablet Weight | 300 | 400 | 600 |
| (Layer 1 + Layer 2) | | | |
| Total Film Coating | 12 | 16 | 24 |

^{†}Salt factor: 1.285 (sitagliptin),
^{‡}Salt factor: 1.034 (atorvastatin)

### Example 4

### Bilayer Tablet Formulations Comprising a 100 mg Sitagliptin Phosphate Layer and an Atorvastatin Anhydrous Lactose (A2) Layer

| | 100/10 | 100/20 | 100/40 | |
|---|---|---|---|---|
| | potency | potency | potency | 100/80 |
| | (mg/ | (mg/ | (mg/ | potency |
| Component | tablet) | tablet) | tablet) | (mg/tablet) |
| Layer 1 | | | | |
| Sitagliptin phosphate^{†} | 128.5 | 128.5 | 128.5 | 128.5 |
| Calcium Phosphate, dibasic | 123.75 | 123.75 | 123.75 | 123.75 |
| Microcrystalline cellulose or | 123.75 | 123.75 | 123.75 | 123.75 |
| silicified Microcrystalline | | | | |
| cellulose | | | | |
| Croscarmellose sodium | 8 | 8 | 8 | 8 |
| Sodium stearyl fumarate | 12 | 12 | 12 | 12 |
| Magnesium stearate | 4 | 4 | 4 | 4 |
| Total Layer 1 weight | 400 | 400 | 400 | 400 |
| Layer 2 | | | | |
| Atorvastatin Calcium^{‡} | 10.34 | 20.68 | 41.36 | 82.72 |
| Microcrystalline cellulose | 47.27 | 94.55 | 189.09 | 378.18 |
| Lactose (anhydrous) | 32.39 | 64.77 | 129.55 | 259.10 |
| Hydroxypropyl cellulose | 3 | 6 | 12 | 24 |
| Croscarmellose sodium | 3 | 6 | 12 | 24 |
| Sodium bicarbonate | 2 | 4 | 8 | 16 |
| Sodium lauryl sulfate | 1 | 2 | 4 | 8 |
| Magnesium stearate | 1 | 2 | 4 | 8 |
| Total Layer 2 weight | 100 | 200 | 400 | 800 |
| Core Bilayer Tablet Weight | 500 | 600 | 800 | 1200 |
| (Layer 1 + Layer 2) | | | | |
| Total Film Coating | 20 | 24 | 32 | 48 |

^{†}Salt factor: 1.285 (sitagliptin),
^{‡}Salt factor: 1.034 (atorvastatin)

### Example 5

### Bilayer Tablet Formulations Comprising a 50 Mg Sitagliptin Phosphate Layer and an Atorvastatin Anhydrous Lactose (A2) Layer

| | 50/10 | 50/20 | 50/40 |
|---|---|---|---|
| | potency | potency | potency |
| Component | (mg/tablet) | (mg/tablet) | (mg/tablet) |
| Layer 1 | | | |
| Sitagliptin phosphate^{†} | 64.25 | 64.25 | 64.25 |
| Calcium Phosphate, dibasic | 61.875 | 61.875 | 61.875 |
| Microcrystalline cellulose | 61.875 | 61.875 | 61.875 |
| or silicified Microcrystalline | | | |
| cellulose | | | |
| Croscarmellose sodium | 4 | 4 | 4 |
| Sodium stearyl fumarate | 6 | 6 | 6 |
| Magnesium stearate | 2 | 2 | 2 |
| Total Layer 1 weight | 200 | 200 | 200 |
| Layer 2 | | | |
| Atorvastatin Calcium^{‡} | 10.34 | 20.68 | 41.36 |
| Microcrystalline cellulose | 47.27 | 94.55 | 189.09 |
| Lactose (anhydrous) | 32.39 | 64.77 | 129.55 |
| Hydroxypropyl cellulose | 3 | 6 | 12 |
| Croscarmellose sodium | 3 | 6 | 12 |
| Sodium bicarbonate | 2 | 4 | 8 |
| Sodium lauryl sulfate | 1 | 2 | 4 |
| Magnesium stearate | 1 | 2 | 4 |
| Total Layer 2 weight | 100 | 200 | 400 |
| Core Bilayer Tablet Weight | 300 | 400 | 600 |
| (Layer 1 + Layer 2) | | | |
| Total Film Coating | 12 | 16 | 24 |

^{†}Salt factor: 1.285 (sitagliptin),
^{‡}Salt factor: 1.034 (atorvastatin)

### Example 6

### Formulations Prepared for Probe Pharmacokinetic Study in Humans (100/80 Potency)

| | Unit Strength (mg) | | | |
|---|---|---|---|---|
| | E1 | E2 | | |
| | For- | For- | | |
| | mula- | mula- | E3 | E4 |
| | tion | tion | Formula- | Formula- |
| | 100/80 | 100/80 | tion | tion |
| Components | mg/ | mg/ | 100/80 | 100/80 |
| Core Tablet: | tablet | tablet | mg/tablet | mg/tablet |
| Layer 1 | | | | |
| Sitagliptin phosphate^{†} | 128.5 | 128.5 | 128.5 | 128.5 |
| Calcium Phosphate, dibasic | 123.75 | 123.75 | 123.75 | 123.75 |
| Microcrystalline cellulose | 123.75 | 123.75 | 123.75 | 123.75 |
| Croscarmellose sodium | 8 | 8 | 8 | 8 |
| Sodium stearyl fumarate | 12 | 12 | 12 | 12 |
| Magnesium stearate | 4 | 4 | 4 | 4 |
| Layer 2 | A1 | A2 | | |
| Atorvastatin calcium^{‡} | 82.72 | 82.72 | 82.72 | 82.72 |
| Microcrystalline cellulose | 296.64 | 378.16 | 296.64 | 296.64 |
| Mannitol | 296.64 | — | 296.64 | 296.64 |
| Lactose (anhydrous) | — | 259.12 | — | — |
| Hydroxypropyl cellulose | 24 | 24 | 24 | 24 |
| Croscarmellose sodium | 48 | 24 | 48 | 48 |
| Sodium bicarbonate | 32 | 16 | 32 | 32 |
| Sodium lauryl sulfate | 8 | 8 | 8 | 8 |
| Magnesium stearate | 12 | 8 | 12 | 12 |
| Core Bilayer Tablet Weight | 1200 | 1200 | 1200 | 1200 |
| (Layer 1 + Layer 2) | | | | |
| Film Coating Suspension: | | | | |
| Sodium | — | — | 39.6 | — |
| carboxymethylcellulose | | | | |
| Film Coat Blend, Powder, | — | — | — | 66.0 |
| Clear [Opaglos ® 2, | | | | |
| 97A19243]^{\|\|} | | | | |
| Sodium bicarbonate | — | — | 6.6 | — |
| Talc | — | — | 19.8 | — |
| Purified water | — | — | § | § |
| Total Tablet Weight (Core + | 1200 | 1200 | 1266 | 1266 |
| Optional Coat) | | | | |

^{†}Salt factor: 1.285 (sitagliptin)
^{‡}Salt factor: 1.034 (atorvastatin)
^{||}Opaglos ® 2 consists of sodium carboxymethylcellulose, maltodextrin, dextrose, and purified stearic acid.
§ Removed during processing.

### Example 7

### Bilayer Tablet Formulation

### General Composition

| | % of | |
|---|---|---|
| | Each | |
| Components | Layer | Range |
| Layer 1 | | |
| Sitagliptin phosphate^{†} | 32.13 | 50 and 100 mg |
| Calcium Phosphate, dibasic | 30.94 | 10-60% (of layer 1) |
| Microcrystalline cellulose or | 30.94 | 10-60% (of layer 1) |
| silicified microcrystalline | | |
| cellulose (Prosolv) | | |
| Croscarmellose sodium | 2.00 | 2-10% (of layer 1) |
| Sodium stearyl fumarate | 3.00 | 0.25-5% (of layer 1) |
| Magnesium stearate | 1.00 | 0.25-5% (of layer 1) |
| Total Layer 1 weight | 100 | |
| Layer 2 | | |
| Atorvastatin Calcium | 10.5 | 10, 20, 40, and 80 mg |
| trihydrate, crystalline^{‡} | | |
| Microcrystalline cellulose | 24.1 | 2-60% (of layer 2) |
| Lactose (monohydrate) | 25.0 | 10-90% (of layer 2) |
| Hydroxypropyl cellulose | 2 | 0-15% (of layer 2) |
| Croscarmellose sodium | 6 | 2-20% (of layer 2) |
| Calcium carbonate | 31 | 2-50% (of layer 2) |
| Polysorbate | 0.4 | 0-5% (of layer 2) |
| Magnesium stearate | 0.5 | 0.1-5% (of layer 2) |
| optional colloidal silicon dioxide | 0 or 0.5 | 0-5% (of layer 2) |
| Total Layer 2 weight | 100 | |
| Core Bilayer Tablet Weight | | |
| (Layer 1 + Layer 2) | | |
| Total Film Coating | 2-5 | 2-10% (of tablet) |

^{†}Salt factor: 1.285 (sitagliptin),
^{‡}Salt factor: 1.082 (atorvastatin).

### Example 8

| | 100/10 | 100/20 | 100/40 | 100/80 |
|---|---|---|---|---|
| | potency | potency | potency | potency |
| | (mg/ | (mg/ | (mg/ | (mg/ |
| Components | tablet) | tablet) | tablet) | tablet) |
| Layer 1 | | | | |
| Sitagliptin phosphate^{†} | 128.5 | 128.5 | 128.5 | 128.5 |
| Calcium Phosphate, dibasic | 123.75 | 123.75 | 123.75 | 123.75 |
| Microcrystalline cellulose or | 123.75 | 123.75 | 123.75 | 123.75 |
| silicified microcrystalline | | | | |
| cellulose (Prosolv) | | | | |
| Croscarmellose sodium | 8 | 8 | 8 | 8 |
| Sodium stearyl fumarate | 12 | 12 | 12 | 12 |
| Magnesium stearate | 4 | 4 | 4 | 4 |
| Total Layer 1 weight | 400 | 400 | 400 | 400 |
| Layer 2 (granulation F1) | | | | |
| Atorvastatin Calcium trihydrate, | 10.82 | 21.64 | 43.28 | 86.56 |
| crystalline^{‡} | | | | |
| Microcrystalline cellulose | 24.83 | 49.67 | 99.34 | 198.68 |
| Lactose monohydrate | 25.76 | 51.52 | 103.05 | 206.10 |
| Hydroxypropyl cellulose | 2.06 | 4.12 | 8.24 | 16.49 |
| Croscarmellose sodium | 6.18 | 12.37 | 24.73 | 49.46 |
| Calcium carbonate | 31.94 | 63.89 | 127.78 | 255.56 |
| Polysorbate 80 | 0.41 | 0.82 | 1.65 | 3.30 |
| Magnesium stearate | 0.52 | 1.03 | 2.06 | 4.12 |
| Colloidal silicon dioxide | 0.52 | 1.03 | 2.06 | 4.12 |
| Total Layer 2 weight | 103.05 | 206.10 | 412.19 | 824.38 |
| Core Bilayer Tablet Weight | 503.05 | 606.10 | 812.19 | 1224.38 |
| (Layer 1 + Layer 2) | | | | |
| Total Film Coating | 20.1 | 24.2 | 32.5 | 49.0 |

^{†}Salt factor: 1.285 (sitagliptin),
^{‡}Salt factor: 1.082 (atorvastatin).
The total film coating is calculated based on 4% weight gain.

### Example 9

| | 50/10 | 50/20 | 50/40 |
|---|---|---|---|
| | potency | potency | potency |
| Components | (mg/tablet) | (mg/tablet) | (mg/tablet) |
| Layer 1 | | | |
| Sitagliptin phosphate^{†} | 64.25 | 64.25 | 64.25 |
| Calcium Phosphate, dibasic | 61.875 | 61.875 | 61.875 |
| Microcrystalline cellulose or | 61.875 | 61.875 | 61.875 |
| silicified microcrystalline | | | |
| cellulose (Prosolv) | | | |
| Croscarmellose sodium | 4 | 4 | 4 |
| Sodium stearyl fumarate | 6 | 6 | 6 |
| Magnesium stearate | 2 | 2 | 2 |
| Total Layer 1 weight | 200 | 200 | 200 |
| Layer 2 (granulation F1) | | | |
| Atorvastatin Calcium | 10.82 | 21.64 | 43.28 |
| trihydrate, crystalline^{‡} | | | |
| Microcrystalline cellulose | 24.83 | 49.67 | 99.34 |
| Lactose monohydrate | 25.76 | 51.52 | 103.05 |
| Hydroxypropyl cellulose | 2.06 | 4.12 | 8.24 |
| Croscarmellose sodium | 6.18 | 12.37 | 24.73 |
| Calcium carbonate | 31.94 | 63.89 | 127.78 |
| Polysorbate 80 | 0.41 | 0.82 | 1.65 |
| Magnesium stearate | 0.52 | 1.03 | 2.06 |
| Colloidal silicon dioxide | 0.52 | 1.03 | 2.06 |
| Total Layer 2 weight | 103.05 | 206.10 | 412.19 |
| Core Bilayer Tablet Weight | 303.05 | 406.10 | 612.19 |
| (Layer 1 + Layer 2) | | | |
| Total Film Coating | 12.1 | 16.2 | 24.5 |

^{†}Salt factor: 1.285 (sitagliptin),
^{‡}Salt factor: 1.082 (atorvastatin).
The total film coating is calculated based on 4% weight gain.

### Example 10

| | 100/10 | 100/20 | 100/40 | 100/80 |
|---|---|---|---|---|
| | potency | potency | potency | potency |
| | (mg/ | (mg/ | (mg/ | (mg/ |
| Components | tablet) | tablet) | tablet) | tablet) |
| Layer 1 | | | | |
| Sitagliptin phosphate^{†} | 128.5 | 128.5 | 128.5 | 128.5 |
| Calcium Phosphate, dibasic | 123.75 | 123.75 | 123.75 | 123.75 |
| Microcrystalline cellulose or | 123.75 | 123.75 | 123.75 | 123.75 |
| silicified microcrystalline | | | | |
| cellulose (Prosolv) | | | | |
| Croscarmellose sodium | 8 | 8 | 8 | 8 |
| Sodium stearyl fumarate | 12 | 12 | 12 | 12 |
| Magnesium stearate | 4 | 4 | 4 | 4 |
| Total Layer 1 weight | 400 | 400 | 400 | 400 |
| Layer 2 (granulation F2) | | | | |
| Atorvastatin Calcium trihydrate, | 10.82 | 21.64 | 43.28 | 86.56 |
| crystalline^{‡} | | | | |
| Microcrystalline cellulose | 24.83 | 49.67 | 99.34 | 198.68 |
| Lactose monohydrate | 25.76 | 51.52 | 103.05 | 206.10 |
| Hydroxypropyl cellulose | 2.06 | 4.12 | 8.24 | 16.49 |
| Croscarmellose sodium | 6.18 | 12.37 | 24.73 | 49.46 |
| Calcium carbonate | 31.94 | 63.89 | 127.78 | 255.56 |
| Polysorbate 80 | 0.41 | 0.82 | 1.65 | 3.30 |
| Magnesium stearate | 0.52 | 1.03 | 2.06 | 4.12 |
| Total Layer 2 weight | 102.53 | 205.06 | 410.13 | 820.26 |
| Core Bilayer Tablet Weight | 502.53 | 605.06 | 810.13 | 1220.26 |
| (Layer 1 + Layer 2) | | | | |
| Total Film Coating | 20.1 | 24.2 | 32.4 | 48.8 |

^{†}Salt factor: 1.285 (sitagliptin),
^{‡}Salt factor: 1.082 (atorvastatin).
The total film coating is calculated based on 4% weight gain.

### Example 11

| | 50/10 | 50/20 | 50/40 |
|---|---|---|---|
| | potency | potency | potency |
| Components | (mg/tablet) | (mg/tablet) | (mg/tablet) |
| Layer 1 | | | |
| Sitagliptin phosphate^{†} | 64.25 | 64.25 | 64.25 |
| Calcium Phosphate, dibasic | 61.875 | 61.875 | 61.875 |
| Microcrystalline cellulose | 61.875 | 61.875 | 61.875 |
| or silicified microcrystalline | | | |
| cellulose (Prosolv) | | | |
| Croscarmellose sodium | 4 | 4 | 4 |
| Sodium stearyl fumarate | 6 | 6 | 6 |
| Magnesium stearate | 2 | 2 | 2 |
| Total Layer 1 weight | 200 | 200 | 200 |
| Layer 2 (granulation F2) | | | |
| Atorvastatin Calcium | 10.82 | 21.64 | 43.28 |
| trihydrate, crystalline^{‡} | | | |
| Microcrystalline cellulose | 24.83 | 49.67 | 99.34 |
| Lactose monohydrate | 25.76 | 51.52 | 103.05 |
| Hydroxypropyl cellulose | 2.06 | 4.12 | 8.24 |
| Croscarmellose sodium | 6.18 | 12.37 | 24.73 |
| Calcium carbonate | 31.94 | 63.89 | 127.78 |
| Polysorbate 80 | 0.41 | 0.82 | 1.65 |
| Magnesium stearate | 0.52 | 1.03 | 2.06 |
| Total Layer 2 weight | 102.53 | 205.06 | 410.13 |
| Core Bilayer Tablet Weight | 302.53 | 405.06 | 610.13 |
| (Layer 1 + Layer 2) | | | |
| Total Film Coating | 12.1 | 16.2 | 24.4 |

^{†}Salt factor: 1.285 (sitagliptin),
^{‡}Salt factor: 1.082 (atorvastatin).
The total film coating is calculated based on 4% weight gain.

### Example 12

### Bilayer Tablet Manufacturing Process

The sitagliptin/atorvastatin bilayer tablet manufacturing process is the same for all formulations. The process consists of the following steps:

Sitagliptin Powder Blend with Microcrystalline Cellulose: Layer 1
1. Sitagliptin phosphate, microcrystalline cellulose or silicified microcrystalline cellulose (such as Avicel 102 or Prosolv®), dibasic calcium phosphate and croscarmellose sodium, and optionally silicon dioxide if Avicel 102, or another Avicel product, is used in place of Proslv®, are blended in a suitable blender.
2. The above blend is lubricated with magnesium stearate and sodium stearyl fumarate in a suitable blender.

### Atorvastatin Granulation (A1 and A2): Layer 2

1. Atorvastatin Calcium, microcrystalline cellulose, either mannitol (A1) or anhydrous lactose (A2), hydroxylpropyl cellulose, croscarmellose sodium, sodium bicarbonate, and sodium lauryl sulfate are first blended in a suitable blender.
2. The blend is de-lumped if necessary.
3. The above blend is lubricated with magnesium stearate in a suitable blender.
4. The lubricated blend is roller compacted and milled into granules using suitable milling equipment.
5. The resulting granules are lubricated with magnesium stearate in a suitable blender.

### Bilayer Tableting

1. The Atorvastatin granulation and Sitagliptin powder blend ate used to compress bilayer tablets in a suitable tablet press.
2. The bilayer tablets are weight sorted using a suitable weight sorting machine if necessary.
3. The bilayer tablets are film coated with two different coating suspensions:
   a. an aqueous suspension consisting of sodium carboxymethylcellulose, sodium bicarbonate, and talc; or
   b. an aqueous Opaglos 2® 97A19243 suspension; or
   c. an aqueous Opadry IT (85F18422)^{&} at the target weight gain of 2-5%. ^{&} Opadry II consists of polyvinyl alcohol, titanium dioxide, macrogol or polyethylene glycol (PEG) 3350, and talc.

### Example 13

### Bilayer tablet manufacturing process

I. Preparation of Sitagliptin with Prosolv® Brand Microcrystalline Cellulose or Sitagliptin with silicified Microcrystalline Cellulose
a. sitagliptin phosphate, microcrystalline cellulose or silicified microcrystalline cellulose (Prosolv), dibasic calcium phosphate and croscarmellose sodium are blended in a suitable blender.
b. The above blend is lubricated with magnesium stearate and sodium stearyl fumarate in a suitable blender.
   II. Preparation of Atorvastatin Granulation with mannitol (A1) and with anhydrous lactose (A2)
a. Atorvastatin Calcium, microcrystalline cellulose, either mannitol (A1) or lactose anhydrous (A2), hydroxylpropyl cellulose, croscarmellose sodium, sodium bicarbonate, and sodium lauryl sulfate are first blended in a suitable blender.
b. The blend is de-lumped if necessary.
c. The above blend is lubricated with magnesium stearate in a suitable blender.
d. The lubricated blend is roller compacted and milled into granules using a suitable milling equipment.
e. The resulting granules are lubricated with magnesium stearate in a suitable blender.
   III. Preparation of coated Bilayer Tablets
a. The atorvastatin granulation (from II) and sitagliptin powder blend (from I) are used to compress bilayer tablets in a suitable tablet press.
b. The bilayer tablets are film coated with an aqueous Opadry II (85F18422)^{&} at the target weight gain of 2-5% ^{&} Opadry II consists of polyvinyl alcohol, titanium dioxide, macrogol or polyethylene glycol (PEG) 3350, and talc.

### Example 14

### Bilayer Tablet Manufacturing Process

I. Preparation of Sitagliptin with Prosolv® Brand Microcrystalline Cellulose or Sitagliptin with silicified Microcrystalline Cellulose
a. Sitagliptin phosphate, microcrystalline cellulose or silicified microcrystalline cellulose (Prosolv), dibasic calcium phosphate and croscarmellose sodium are blended in a suitable blender.
b. The above blend is lubricated with magnesium stearate and sodium stearyl fumarate in a suitable blender.

### II. Preparation of Atorvastatin Granulations (F1 and F2)

a. The granulating solution was prepared by adding hydroxylpropyl cellulose in purified water until all solids dissolved.
b. Polysorbate 80 was then added and agitated until the solution became clear.
c. Atorvastatin, microcrystalline cellulose, lactose monohydrate, croscarmellose sodium, and calcium carbonate are granulated in a top-spray fluidized bed column with the granulating solution, followed by drying in the same fluidized bed column.
d. The dried granules were milled using a suitable milling equipment.
e. The milled granules were blended with colloidal silicon dioxide and lubricated with magnesium stearate in a suitable blender. The final blend is the Atorvastatin granulation with silicon dioxide (granulation F1); or
f. Alternatively, the milled granules were lubricated with magnesium stearate in a suitable blender. The final blend is the Atorvastatin granulation without silicon dioxide (granulation F2).
   III. Preparation of coated Bilayer Tablets
a. The atorvastatin granulation (from II either e or f) and sitagliptin powder blend (from I) are used to compress bilayer tablets in a suitable tablet press.
b. The bilayer tablets are film coated with an aqueous Opadry II (85F)^{&} at the target weight gain of 2-5%. ^{&} Opadry II consists of polyvinyl alcohol, titanium dioxide, macrogol or polyethylene glycol (PEG) 3350, talc and, if applicable, iron oxide.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims which follow and that such claims be interpreted as broadly as is reasonable.

1. A pharmaceutical composition in the form of a bilayer tablet comprising:
(a) a first layer comprising about 20 to 45% by weight of a dipeptidyl peptidase-4 inhibitor, or a pharmaceutically acceptable salt thereof; and
(b) a second layer comprising about 5 to 15% by weight of atorvastatin, or a pharmaceutically acceptable salt thereof.

2. (canceled)

3. The pharmaceutical composition of claim 1 wherein the dipeptidyl peptidase-4 inhibitor is sitagliptin, or the dihydrogenphosphate salt thereof.

4. The pharmaceutical composition of claim 1 wherein the second layer additionally comprises 0-4% of an alkalizing agent selected from the group consisting of L-arginine and sodium bicarbonate.

5. (canceled)

6. The pharmaceutical composition of claim 1 wherein the second layer further comprises 2 to 50% of an alkalizing agent.

7. The pharmaceutical composition of claim 6 wherein said alkalizing agent is calcium carbonate.

8. (canceled)

9. (canceled)

10. (canceled)

11. (canceled)

12. The pharmaceutical composition of claim 1 comprising:
(a) a first layer comprising:
(i) about 20 to 45% by weight of a dipeptidyl peptidase-4 inhibitor, or a pharmaceutically acceptable salt thereof;
(ii) about 40 to 80% by weight of a diluent;
(iii) about 0.1 to 10% by weight of a disintegrant; and
(iv) about 0.5 to 10% by weight of a lubricant; and
(b) a second layer comprising:
(i) about 5 to 15% by weight of atorvastatin, or a pharmaceutically acceptable salt thereof;
(ii) about 2 to 90% by weight of a diluent;
(iii) about 0 to 15% of a binding agent;
(iv) about 0.1 to 20% by weight of a disintegrant;
(v) about 0 to 5% by weight of a surfactant, and
(vi) about 0.25 to 5% by weight of a lubricant.

13. The pharmaceutical composition of claim 12 wherein the first layer further comprises a glidant.

14. The pharmaceutical composition of claim 13 wherein the glidant is silicon dioxide.

15. The pharmaceutical composition of claim 12 wherein the diluent in the first layer is selected from the group consisting of: microcrystalline cellulose, mannitol and anhydrous dibasic calcium phosphate, or a mixture thereof; the disintegrant is selected from the group consisting of: crospovidone and croscarmellose sodium, or a mixture thereof; and the lubricant is selected from the group consisting of: magnesium stearate and sodium stearyl fumarate, or a mixture thereof.

16. (canceled)

17. The pharmaceutical composition of claim 12 wherein the diluent in the second layer is selected from the group consisting of: microcrystalline cellulose, anhydrous lactose and mannitol, or a mixture thereof; the binding agent is hydroxypropyl cellulose; the disintegrant is croscarmellose sodium; the surfactant is sodium lauryl sulfate; and the lubricant is selected from the group consisting of: magnesium stearate, and sodium stearyl fumarate, or a mixture thereof.

18. (canceled)

19. (canceled)

20. The pharmaceutical composition of claim 12 wherein the second layer further comprises 2 to 50% of an alkalizing agent.

21. The pharmaceutical composition of claim 20 wherein the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate, and the alkalizing agent is calcium carbonate.

22. The pharmaceutical composition of claim 12 wherein the second layer further comprises 2 to 50% of an alkalizing agent and 0.1 to 5% of a glidant.

23. The pharmaceutical composition of claim 22 wherein the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate, the alkalizing agent is calcium carbonate, and the glidant is silicone dioxide.

24. The pharmaceutical composition of claim 1, wherein said pharmaceutically acceptable salt of atorvastatin is selected from the group consisting of a calcium salt, a sodium salt, and a magnesium salt, or a hydrate or solvate thereof.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The pharmaceutical composition of claim 1 comprising
(a) a first layer comprising:
(i) about 20 to 45% by weight of a dipeptidyl peptidase-4 inhibitor, or a pharmaceutically acceptable salt thereof;
(ii) about 40 to 80% by weight of a diluent;
(iii) about 0.5 to 10% by weight of a disintegrant; and
(iv) about 0.5 to 10% by weight of a lubricant; and
(b) a second layer comprising:
(i) about 5 to 15% by weight of atorvastatin;
(ii) about 30 to 70% by weight of a diluent;
(iii) about 0.1 to 15% of a binding agent;
(iv) about 2 to 20% by weight of a disintegrant;
(v) about 0.1 to 5% by weight of a surfactant, and
(vi) about 0.1 to 5% by weight of a lubricant.

38. The pharmaceutical composition of claim 37 wherein the diluent in the first layer is a mixture of anhydrous dibasic calcium phosphate and microcrystalline cellulose or silicified microcrystalline cellulose; the disintegrant is croscarmellose sodium; and the lubricant is a mixture of sodium stearyl fumarate and magnesium stearate.

39. The pharmaceutical composition of claim 37 wherein the second layer further comprises 2 to 50% of an alkalizing agent.

40. The pharmaceutical composition of claim 39 wherein the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate, and the alkalizing agent is calcium carbonate.

41. The pharmaceutical composition of claim 37 wherein the second layer further comprises 2 to 50% of an alkalizing agent and 0.1 to 5% of a glidant.

42. The pharmaceutical composition of claim 41 wherein the diluent in the second layer is a mixture of microcrystalline cellulose and lactose monohydrate; the binding agent is hydroxypropyl cellulose; the disintegrant is croscarmellose sodium; the surfactant is polysorbate 80; the lubricant is magnesium stearate, the alkalizing agent is calcium carbonate, and the glidant is silicone dioxide.

43. The pharmaceutical composition of claim 1 wherein the dipeptidyl peptidase-4 inhibitor is present in a unit dosage strength of 25, 50, 75, 100, 150, or 200 milligrams, and the atorvastatin is present in a unit dosage strength of 5, 10, 20, 40 or 80 milligrams.

44. The pharmaceutical composition of claim 1 wherein the dipeptidyl peptidase-4 inhibitor is sitagliptin, or a pharmaceutically acceptable salt thereof.

45. (canceled)

46. (canceled)

47. The pharmaceutical composition of claim 1, wherein said pharmaceutically acceptable salt of atorvastatin is selected from the group consisting of a calcium salt, a sodium salt, and a magnesium salt, or a hydrate or solvate thereof.

48. (canceled)

49. (canceled)

50. (canceled)

51. (canceled)

52. A method of treating Type 2 diabetes in a human in need thereof comprising orally administering to said human a pharmaceutical composition of claim 1.

53. (canceled)

54. (canceled)
